(19) United States
(12) Patent Application Publication
(10) Pub. No.: US 2026/0199423 A1
(43) Pub. Date: Jul. 16, 2026

(54) COMPOSITIONS COMPRISING AN ONCOLYTIC PEPTIDE AND CHITOSAN

(71) Applicant: LYTIX BIOPHARMA AS, Oslo (NO)

(72) Inventors: Gry Stensrud, Nittedal (NO); Baldur Sveinbjörnsson, Oslo (NO); Raul Perez Gonzalez, Vitoria-Gasteiz (ES); Oihane Gartziandia Lopez de Goikoetxea, Olazti (ES)

(21) Appl. No.: 19/137,520

(22) PCT Filed: Dec. 20, 2023
(86) PCT No.: PCT/EP2023/087135

§ 371 Date: Jun. 10, 2025

Related U.S. Application Data
(60) Provisional application No. 63/476,228, filed on Dec. 20, 2022.

(30) Foreign Application Priority Data
Jan. 30, 2023 (EP) 23153990.9
Mar. 14, 2023 (NO) 20230275

(51) Int. Cl. A61K 38/08; A61K 9/00; A61K 31/205; A61K 47/36; A61P 35/00

(52) U.S. Cl. CPC A61K 38/08; A61K 9/0019; A61K 31/205; A61K 47/36; A61P 35/00

(57) ABSTRACT

The disclosure herein relates to compositions, kits and their use for treating a subject, e.g. a patient, having a tumor by administering to the subject such compositions.

### TECHNICAL FIELD

The disclosure herein relates to compositions, kits and their use for treating a subject, e.g. a patient, having a tumor by administering to the subject such compositions.

### BACKGROUND

Oncolytic peptides are highly effective anticancer agents. Not only do these peptides have a direct lytic action on tumor cells through disturbing and permeabilizing the cell membrane, they are also highly effective at attacking organelles such as mitochondria and lysosomes and can cause lysis thereof. This disruption of the organelle membrane results in the release of agents therefrom which have a potent immunostimulatory function, such agents are generally known as damage-associated molecular pattern molecules (DAMPs) and include ATP, cytochrome C, mitochondrial CpG DNA sequences, mitochondrial formyl peptides, cathepsins (from lysosomes) and HMGB1 (from the nucleus). As a consequence, these oncolytic peptides stimulate the immune system and elicit an immune response which generates long term antitumor immunity. The immune stimulation, which occurs at the time the DAMPs and the tumor antigens are released by injecting an oncolytic peptide into a tumor and its direct lytic effect on said tumor, results in enhanced antigen presentation and thus a type of in situ “vaccination” against the tumor.

While oncolytic peptides are a promising tool in cancer immunotherapy, there is still room for improving their use in cancer therapy.

### SUMMARY

In a first aspect, the present disclosure relates to a combination of a composition (a) comprising an oncolytic peptide and a composition (b) comprising chitosan and/or a chitosan derivative for use in the treatment of a subject having a tumor or relates to a composition (a) comprising an oncolytic peptide and a composition (b) comprising chitosan and/or a chitosan derivative for use in the treatment of a subject having a tumor.

In some embodiments, compositions (a) and (b) are used in the treatment concurrently. In some other embodiments, compositions (a) and (b) are used in the treatment sequentially. In yet some other embodiments, compositions (a) and (b) are used in the treatment combined into one composition, i.e. a composition comprising i) an oncolytic peptide and ii) chitosan and/or a chitosan derivative.

Thus, in some embodiments, the first aspect of the present disclosure relates to a combination of a composition (a) comprising an oncolytic peptide and a composition (b) comprising chitosan and/or a chitosan derivative for concurrent, sequential or combined use in the treatment of a subject having a tumor or relates to a composition (a) comprising an oncolytic peptide and a composition (b) comprising chitosan and/or a chitosan derivative for concurrent, sequential or combined use in the treatment of a subject having a tumor, e.g., wherein compositions (a) and (b) are administered to said subject concurrently, sequentially or as a combined composition.

In an alternative first aspect, the present disclosure relates to a composition (a) which comprises an oncolytic peptide for use in the treatment of a subject having a tumor by concurrent, sequential or combined administration with a composition (b) which comprises chitosan and/or a chitosan derivative.

In yet an alternative first aspect, the present disclosure relates to a method of treating a subject having a tumor comprising the concurrent, sequential or combined administration to said subject of a composition (a) comprising an oncolytic peptide and a composition (b) comprising chitosan and/or a chitosan derivative.

In yet an alternative first aspect, the present disclosure relates to the use of composition (a) comprising an oncolytic peptide and a composition (b) comprising chitosan and/or a chitosan derivative for treating a subject having a tumor. In some embodiments, compositions (a) and (b) are used concurrently, sequentially or as a combined composition for treating said subject, e.g. compositions (a) and (b) are administered to said subject concurrently, sequentially or as a combined composition.

In some embodiments, the aforementioned compositions comprise a physiologically acceptable carrier. In some embodiments, composition (a) is administered into said tumor and composition (b) is administered into the tumor and/or the tissue immediately surrounding the tumor.

In some embodiments, the afore-mentioned compositions are pharmaceutical compositions.

In some embodiments, the composition comprising the oncolytic peptide and the composition comprising chitosan and/or a chitosan derivative are combined into one composition.

Thus, in some embodiments, the disclosure relates to a composition comprising i) an oncolytic peptide and ii) chitosan and/or a chitosan derivative for use in the treatment of a subject having a tumor. In some embodiments, the composition is administered to said subject, e.g. is administered into the tumor of said subject.

Alternatively, the disclosure relates to a method of treating a subject having a tumor comprising administering to said subject a composition comprising i) an oncolytic peptide and ii) chitosan and/or a chitosan derivative. In some embodiments, the composition is administered into the tumor.

In another alternative, the disclosure relates to the use of i) an oncolytic peptide and ii) chitosan and/or a chitosan derivative for the manufacture of a medicament for use in the treatment of a subject having a tumor. In some embodiments, said medicament is administered to said subject, e.g. is administered into the tumor of said subject.

In yet another alternative, the disclosure relates to a composition comprising i) an oncolytic peptide and ii) chitosan and/or a chitosan derivative for treating a subject having a tumor. In some embodiments, the composition is administered to said subject, e.g. is administered into the tumor of said subject.

In yet another alternative, the disclosure relates to a medicament for the treatment of a tumor in a subject by administering said medicament to said subject, wherein the medicament comprises i) an oncolytic peptide and ii) chitosan and/or a chitosan derivative. In some embodiments, said medicament is administered into the tumor.

In some embodiments, the afore-mentioned composition comprises a physiologically acceptable carrier. In some embodiments, the afore-mentioned composition is a pharmaceutical composition.

In another aspect, the present disclosure relates to a composition comprising i) an oncolytic peptide and ii) chitosan and/or a chitosan derivative.

In some embodiments, said composition comprises a physiologically acceptable carrier.

In some embodiments, said composition is a pharmaceutical composition.

In yet another aspect, the present disclosure relates to a composition comprising i) an oncolytic peptide and ii) chitosan and/or a chitosan derivative thereof for use as a medicament.

In some embodiments, said composition comprises a physiologically acceptable carrier.

In some embodiments, said composition is a pharmaceutical composition.

In yet another aspect, the present disclosure relates to a method for preparing a composition comprising i) an oncolytic peptide and ii) chitosan and/or a chitosan derivative.

In some embodiments, said composition comprises a physiologically acceptable carrier and/or is sealed in a container.

In some embodiments, said composition is a pharmaceutical composition.

In yet another aspect, the present disclosure relates to a kit comprising a composition (a) comprising an oncolytic peptide and a composition (b) comprising chitosan and/or a chitosan derivative or comprising parts/components to prepare compositions (a) and (b.

In yet another aspect, the present disclosure relates to a kit comprising parts/components to prepare a composition comprising i) an oncolytic peptide and ii) chitosan and/or a chitosan derivative.

### DESCRIPTION OF THE DRAWINGS

FIG. 1 shows the results of Example 3, i.e. the therapeutic effect of compositions comprising LTX-401 and chitosan in mice having a tumor, compared to compositions comprising LTX-401 alone, LTX-401 and HPMC or control. Therapeutic efficacy is shown as % of mice having a tumor volume of less than 1 ml/days since treatment start.

FIG. 2 shows the results of Example 4, i.e. the therapeutic effect of compositions comprising LTX-401 and chitosan in mice having a tumor, compared to compositions comprising LTX-401 alone, chitosan alone or control. Therapeutic efficacy is shown as % survival/days post tumor challenge.

### DETAILED DESCRIPTION

A “subject” is an animal, e.g. a mouse, or a human, preferably a human. A subject may be a patient, i.e. a human suffering from cancer who is in need of a treatment of the cancer or who is in need of a treatment to prevent the recurrence of the cancer. The terms “subject” and “individual” are used interchangeably herein.

A “cancer” as used herein refers to a broad group of various diseases characterized by the uncontrolled growth of abnormal cells in the body resulting in a tumor. A “tumor” or “cancer tissue” or “(cancerous) lesion” include a primary tumor and metastases. A tumor includes a solid and non-solid tumor.

A “treatment” as used herein is a therapeutic treatment.

A “therapeutic treatment” is a treatment administered to a subject who displays symptoms or signs of cancer, in which treatment is administered to the subject for the purpose of diminishing or eliminating those signs or symptoms or for the purpose of delaying or stopping disease progression, such as stopping or delaying the growth of a tumor or its increase in malignancy or its spread.

The term “oncolytic peptide” as used herein denotes oncolytic, positively charged, amphipathic amino acid derivatives, peptides or peptidomimetics, i.e. positively charged, amphipathic amino acid derivatives, peptides or peptidomimetics which are able to lyse tumor cell membranes. Being amphipathic in nature, they have one or more hydrophilic groups, i.e. cationic groups, and one or more hydrophobic groups. Thus, these molecules are a) attracted to the negative charge of tumor cell membranes and b) able to interact with the hydrophobic tail of the phospholipids comprised in the tumor cell membrane, which tail consists of fatty acid chains, with their hydrophobic group(s).

### Oncolytic Peptide

As mentioned above, the oncolytic peptide for use in the present disclosure denotes oncolytic positively charged amphipathic amino acid derivatives, peptides or peptidomimetics. Oncolytic peptides have been described in patent publications WO 2007/107748 A2, WO 2010/060497 A1, WO 2011/051692 A1, WO 2016/091487 A1 and WO 2016/091490 A1, the content of which is incorporated herein in its entirety.

### 9-Mer Peptides

In some embodiments, the oncolytic peptide for use in the present disclosure is a 9-mer peptide or a pharmaceutically acceptable salt thereof, which consists of 9 amino acids in a linear arrangement, wherein of those 9 amino acids, 5 are cationic and 4 are lipophilic, and wherein of these 4 lipophilic amino acids, 3 are tryptophan and 1 is a non-genetically coded amino acid.

In some embodiments, the in 9-mer peptide as described above, the lipophilic and cationic amino acids are arranged such that there are no more than two of either type of amino acid adjacent to one another and/or the peptide comprises two pairs of adjacent cationic amino acids and one or two pairs of adjacent lipophilic amino acids.

The term “amino acid” as used for the definition of the 9-mer peptide includes a, β and γ amino acids as well as N substituted glycines.

A “non-genetically coded amino acid” is an amino acid that is structurally, not just stereospecifically, different from the genetically coded L amino acids. As disclosed herein, a D amino acid, while not strictly genetically coded, is thus not considered to be a non-genetically coded amino acid.

The cationic amino acids may be the same or different. In some embodiments, the cationic amino acids are lysine and/or arginine. In some other embodiments, the cationic amino acids are histidine and/or a non-genetically coded amino acid carrying a positive charge at pH 7.0. Lysine and arginine are preferred cationic amino acids.

Non-genetically coded cationic amino acids include derivatives of lysine, arginine and histidine such as homolysine, ornithine, diaminobutyric acid, diaminopimelic acid, diaminopropionic acid, homoarginine, trimethylysine, trimethylornithine, 4-aminopiperidine-4-carboxylic acid, 4-amino-1-carbamimidoylpiperidine-4-carboxylic acid and 4-guanidinophenylalanine.

The non-genetically coded lipophilic amino acids may be the same or different.

In some embodiments, the non-genetically coded lipophilic amino acid has a lipophilic side chain with at least 7, preferably at least 8 or 9, more preferably at least 10 non-hydrogen atoms, preferably carbon atoms. In some embodiments, the lipophilic side chain contains no more than 30 non hydrogen atoms, more preferably no more than 25 non hydrogen atoms. In some embodiments, the lipophilic side chain comprises at least one, preferably two cyclic groups, which may be fused or connected.

In some embodiments, the lipophilic side chain contains heteroatoms such as O, N or S but typically there is no more than one heteroatom, preferably it is nitrogen. The lipophilic side chain will preferably have no more than 2 polar groups, more preferably none or one, most preferably none.

Preferred non-genetically coded lipophilic amino acids include: 2-amino-3-(biphenyl-4-yl) propanoic acid (biphenylalanine), 2-amino-3,3-diphenylpropanoic acid (diphenylalanine), 2-amino-3-(anthracen-9-yl) propanoic acid, 2-amino-3-(naphthalen-2-yl) propanoic acid, 2-amino-3-(naphthalen-1-yl) propanoic acid, 2-amino-3-[1,1′: 4′,1″-terphenyl-4-yl]-propionic acid, 2-amino-3-(2,5,7-tri-tert-butyl-1H-indol-3-yl) propanoic acid, 2-amino-3-[1,1′: 3′,1″-terphenyl-4-yl]-propionic acid, 2-amino-3-[1,1′: 2′,1″-terphenyl-4-yl]-propionic acid, 2-amino-3-(4-naphthalen-2-yl-phenyl)-propionic acid, 2-amino-3-(4′-butylbiphenyl-4-yl) propanoic acid, 2-amino-3-[1,1′: 3′,1″-terphenyl-5′-yl]-propionic acid and 2-amino-3-(4-(2,2-diphenylethyl)phenyl) propanoic acid. Diphenylalanine and biphenylalanine are especially preferred non-genetically coded lipophilic amino acids.

In some embodiments, the aforementioned peptide may have some or all of the amino acids present in it in the D form.

In a preferred embodiment the 9-mer peptide is a compound of formulae (I) to (V) listed below, in which C represents a cationic amino acid as defined above and L represents a lipophilic amino acid as defined above. The amino acids are covalently linked by peptide bonds. The free amino or carboxy terminals of these molecules may be modified, the carboxy terminus is preferably modified to remove its negative charge, most preferably the carboxy terminus is amidated, and this amide group may be substituted.

(SEQ ID NO: 1)
CCLLCCLLC (I)
(SEQ ID NO: 2)
LCCLLCCLC (II)
(SEQ ID NO: 3)
CLLCCLLCC (III)
(SEQ ID NO: 4)
CCLLCLLCC (IV)
(SEQ ID NO: 5)
CLCCLLCCL (V)

As discussed above, the peptide comprises one non-genetically coded lipophilic amino acid. When this amino acid is denoted L′, preferred peptides are represented by the following formulae:

(SEQ ID NO: 6)
CCL'LCCLLC (I')
(SEQ ID NO: 7)
CCLLCCLL'C (I″)
(SEQ ID NO: 8)
CCLL'CCLLC (I″)
(SEQ ID NO: 9)
LCCLL'CCLC (II')

Particularly preferred are the 9-mer peptides of formula (I) and (II), and of these, peptides of formula (I″) are especially preferred.

The 9-mer peptides listed in table below
are most preferred:

| | SEQ | |
|---|---|---|
| | ID | |
| Name | NO | Sequence |
| LTX-301 | 10 | Dip-K-K-W-W-K-K-W-K-NH₂ |
| LTX-302 | 11 | W-K-K-W-Dip-K-K-W-K-NH₂ |
| LTX-303 | 12 | W-K-K-W-W-K-K-Dip-K-NH₂ |
| LTX-304 | 13 | Bip-K-K-W-W-K-K-W-K-NHₛ |
| LTX-305 | 14 | W-K-K-Bip-W-K-K-W-K-NH₂ |
| LTX-306 | 15 | w-k-k-w-dip-k-k-w-k-NH₂ |
| LTX-307 | 16 | K-K-W-Dip-K-K-W-W-K-NH₂ |
| LTX-308 | 17 | k-k-W-Dip-k-k-W-W-k-NH₂ |
| LTX-309 | 18 | K-K-W-Dip-K-K-W-Dip-K-NH₂ |
| LTX-310 | 19 | K-K-W-Bip-K-K-W-W-K-NH₂ |
| LTX-312 | 20 | K-Bip-K-K-W-W-K-K-W-NH₂ |
| LTX-313 | 21 | K-K-Bip-W-K-K-W-W-K-NH₂ |
| LTX-314 | 22 | K-K-W-W-K-K-Dip-W-K-NH₂ |
| LTX-315 | 23 | K-K-W-W-K-K-W-Dip-K-NH₂ |
| LTX-316 | 24 | K-W-Dip-K-K-W-W-K-K-NH₂ |
| LTX-317 | 25 | K-K-W-W-K-W-Dip-K-K-NH₂ |
| LTX-318 | 26 | Orn-Orn-W-Dip-Orn-Orn-W-W-Orn-NH₂ |
| LTX-319 | 27 | Dap-Dap-W-Dip-Dap-Dap-W-W-Dap-NH₂ |
| LTX-320 | 28 | R-R-W-Dip-R-R-W-W-R-NH₂ |
| LTX-321 | 29 | K-W-W-K-K-Dip-W-K-K-NH₂ |
| LTX-323 | 30 | K-Dip-K-K-W-W-K-K-W-NH₂ |
| LTX-324 | 31 | K-K-Dip-W-K-K-W-W-K-NH₂ |
| LTX-325 | 32 | k-w-w-k-k-dip-w-k-k-NH₂ |
| LTX-326 | 33 | R-R-Bip-W-R-R-W-W-R-NH₂ |
| LTX-327 | 34 | R-R-Dip-W-R-R-W-W-R-NH₂ |
| LTX-329 | 35 | k-k-bip-w-k-k-w-w-k-NH₂ |
| LTX-331 | 36 | k-k-Bip-w-k-k-w-w-k-NH₂ |
| LTX-332 | 37 | K-K-bip-W-K-K-W-W-K-NH₂ |
| LTX-333 | 38 | Dab-Dab-W-Dip-Dab-Dab-W-W-Dab-NH₂ |
| LTX-334 | 39 | K-K-W-1-Nal-K-K-W-W-K-NH₂ |
| LTX-335 | 40 | K-K-W-2-Nal-K-K-W-W-K-NH₂ |
| LTX-336 | 41 | K-K-W-Ath-K-K-W-W-K-NH₂ |
| LTX-338 | 42 | K-K-W-Phe(4-4'Bip)-K-K-W-W-K-NH₂ |

in which:
the standard single letter code is used for the genetically coded amino acids
lower case denotes D amino acids
Dip is diphenylalanine
Bip is biphenylalanine
Orn is ornithine
Dap is 2,3-diaminopropionic acid
Dab is 2,4-diaminobutyric acid
1-Nal is 1-naphthylalanine
2-Nal is 2-naphthylalanine
Ath is 2-amino-3-(anthracen-9-yl) propanoic acid
Phe (4,4′Bip) is 2-amino-3-[1,1′: 4′,1″-terphenyl-4-yl]propionic acid

Preferred 9-mer peptides are LTX-302, LTX-313, LTX-315, LTX-320 and LTX-329. Said preferred peptides are preferably in the form of a pharmaceutically acceptable salt, preferably in the form of an acetate salt.

For use in the invention, all of the 9-mer peptides described herein may be in the form of a pharmaceutically acceptable salt. The peptides preferably have a modified, particularly an amidated, C-terminus. Suitable pharmaceutically acceptable salts are well known in the art and include salts of inorganic or organic acids, and include hydrochloride, trifluoroacetate and acetate salts. Acetate salts are most preferred.

Especially preferred is the 9-mer peptide known as LTX-315, in particular in the form of a pharmaceutically acceptable salt, preferably in the form of its acetate salt, e.g. LTX-315 having the amino acid sequence of SEQ ID NO: 23, or a pharmaceutically acceptable salt thereof, preferably the acetate salt thereof.

The 9-mer peptide for use in the disclosure of the invention and its pharmaceutically acceptable salts may be synthesised in any convenient way by methods of peptide synthesis well known in the art, preferably by carrying out the synthesis on a solid phase support. Methods of synthesis are for example described in WO 2010/060497 A1, WO 2016/091487 A1 and WO 2016/091490 A1.

### Compounds Comprising a Disubstituted β Amino Acid

In some embodiments, the oncolytic peptide for use in the present disclosure is a peptide, peptidomimetic or amino acid derivative having a net positive charge of at least +2 and incorporating a disubstituted β amino acid, each of the substituting groups in the β amino acid, which may be the same or different, comprises at least 7 non-hydrogen atoms, is lipophilic and has at least one cyclic group, one or more cyclic groups within a substituting group may be linked or fused to one or more cyclic groups within the other substituting group and where cyclic groups are fused in this way the combined total number of non-hydrogen atoms for the two substituting groups is at least 12. The 2 substituting groups on the β amino acid are preferably the same.

A β amino acid has the amino group attached to the β carbon atom; genetically coded amino acids are α amino acids in which the amino group is attached to the α carbon atom. This arrangement lengthens by one atom per β amino acid the backbone of a peptide incorporating one or more β amino acids. In this arrangement the α and/or the β carbon atom can be substituted. The α or β carbon atom may be disubstituted; where the α carbon atom is disubstituted a β^{2,2} amino acid results and where the β carbon atom is disubstituted a β^{3,3} amino acid is generated. One substituting group on each of the α or β carbon atoms results in a β^{2,3} amino acid. β^{2,2} and β^{3,3} disubstituted amino acids are preferred for use in accordance with the invention, β^{2,2} disubstituted amino acids being especially preferred.

The β amino acid is substituted by two groups incorporating at least 7 non-hydrogen atoms. Preferably one, more preferably both of the substituting groups contains at least 8, more preferably at least 10 non-hydrogen atoms. These groups are lipophilic in nature and while they may be different, are preferably the same. Each contains at least one cyclic group, typically a 6-membered ring which may be aliphatic or aromatic, preferably aromatic, and may be substituted, substituting groups may include hetero atoms such as oxygen, nitrogen, sulphur or a halogen, in particular fluorine or chlorine. Preferred substituting groups include C₁-C₄ alkyl (especially t-butyl), methoxy, fluoro and fluoromethyl groups. The cyclic groups may be homo- or heterocyclic, preferably they are homocyclic ring of carbon atoms. Preferred lipophilic substituting groups incorporate two or three cyclic groups, preferably two cyclic groups, which may be connected or fused, preferably fused. Particularly preferred substituting groups comprise a naphthalene group.

A further preferred group of lipophilic substituting groups have a single substituted or unsubstituted cyclic group, preferably a phenyl or cyclohexyl group.

The cyclic group or groups is typically spaced away from the peptide backbone (i.e. from the α or β carbon atom of the β amino acid) by a chain of 1 to 4, preferably 1 to 3 atoms; these linking atoms may include nitrogen and/or oxygen but will typically be carbon atoms, preferably the linking atoms are unsubstituted. These spacers are of course part of the substituting groups as defined herein.

Each substituting moiety of the disubstituted β amino acid will typically comprise 7 to 20 non-hydrogen atoms, preferably 7 to 13, more preferably 8 to 12, most preferably 9-11 non-hydrogen atoms.

The compounds comprising a disubstituted β amino acid are preferably peptides or peptidomimetics of 1 or 2 to 12 amino acids or equivalent subunits in length. Unless otherwise clear from the context, reference herein to ‘amino acids’ includes the equivalent subunit in a peptidomimetic. The preferred compounds comprise 3 to 12 amino acids, more preferably 5 to 12 amino acids in length. In some embodiments, the compounds comprising a disubstituted β amino acid consists of such disubstituted β amino acid, i.e. is an amino acid derivative (i.e. a modified amino acid).

The compounds comprising a disubstituted β amino acid preferably incorporate a modified C terminus, the C terminal modifying group typically resulting in charge reversal, i.e. removing the negative charge of the carboxyl group and adding a positive charge, e.g. through the presence of an amino group. This modification alone, assuming the N terminus is not modified, will give the molecule overall a net charge of +2. Whether the C terminus is modified to give charge reversal or simply to remove the negative charge of the carboxyl group, in some embodiments wherein the compounds are peptides or peptidomimetics, such compounds also contain one or more cationic amino acids. Thus the overall charge of the compounds may be +3, +4 or higher.

Suitable C-terminal groups, which are preferably cationic in nature, will typically have a maximum size of 15 non-hydrogen atoms. The C-terminus is preferably amidated and the amide group may be further substituted to form an N-alkyl or N, N-dialkyl amide. Primary and secondary amide groups are preferred. Suitable groups to substitute the amide group include aminoalkyl, e.g. amino ethyl or dimethylaminoethyl; the nitrogen atom of the amide group may form part of a cyclic group e.g. pyrazolidine, piperidine, imidazolidine and piperazine, piperazine being preferred, these cyclic groups may themselves be substituted, for example by alkyl or aminoalkyl groups.

In some embodiments, the compounds comprising a disubstituted β amino acid are peptides which incorporate one or more cationic amino acids. Lysine, arginine, ornithine and histidine are preferred but any non-genetically coded or modified amino acid carrying a positive charge at pH 7.0 may be incorporated.

Suitable non-genetically coded cationic amino acids and modified cationic amino acids include analogues of lysine, arginine and histidine such as homolysine, ornithine, diaminobutyric acid, diaminopimelic acid, diaminopropionic acid and homoarginine as well as trimethylysine and trimethylornithine, 4-aminopiperidine-4-carboxylic acid, 4-amino-1-carbamimidoylpiperidine-4-carboxylic acid and 4-guanidinophenylalanine.

In some embodiments, the compounds comprising a disubstituted β amino acid are dipeptides which will typically incorporate one cationic amino acid. Longer peptides will usually incorporate additional cationic amino acids, thus a peptide of 4 or 5 amino acids may have 2 or 3 cationic amino acids and peptides of 6 to 9 amino acids may have 3 to 6 cationic amino acids.

In some embodiments, the compounds comprising a disubstituted β amino acid comprise a β^{2,2} disubstituted amino acid coupled to a C-terminal L-arginine amide residue and dipeptides having this arrangement are preferred.

Peptides with three or more amino acids will typically have one or more additional lipophilic amino acids, i.e., amino acids with a lipophilic R group. Typically the lipophilic R group has at least one, preferably two cyclic groups, which may be fused or connected. The lipophilic R group may contain hetero atoms such as O, N or S but typically there is no more than one heteroatom, preferably it is nitrogen. This R group will preferably have no more than 2 polar groups, more preferably none or one, most preferably none.

Tryptophan is a preferred lipophilic amino acid and peptides preferably comprise 1 to 3 tryptophan residues. Further genetically coded lipophilic amino acids which may be incorporated are phenylalanine and tyrosine.

The lipophilic amino acids may be non-genetically coded, including genetically coded amino acids with modified R groups.

A peptidomimetic is typically characterised by retaining the polarity, three dimensional size and functionality (bioactivity) of its peptide equivalent but wherein the peptide bonds have been replaced, often by more stable linkages. By ‘stable’ is meant more resistant to enzymatic degradation by hydrolytic enzymes. Generally, the bond which replaces the amide bond (amide bond surrogate) conserves many of the properties of the amide bond, e.g. conformation, steric bulk, electrostatic character, possibility for hydrogen bonding etc. Chapter 14 of “Drug Design and Development”, Krogsgaard, Larsen, Liljefors and Madsen (Eds) 1996, Horwood Acad. Pub provides a general discussion of techniques for the design and synthesis of peptidomimetics. In the present disclosure, where the oncolytic peptide/compounds comprising a disubstituted β amino acid is reacting with a tumor cell membrane rather than the specific active site of an enzyme, some of the problems described of exactly mimicking affinity and efficacy or substrate function are not relevant and a peptidomimetic can be readily prepared based on a given peptide structure or a motif of required functional groups. Suitable amide bond surrogates include the following groups: N-alkylation (Schmidt, R. et al., Int. J. Peptide Protein Res., 1995, 46,47), retro-inverse amide (Chorev, M and Goodman, M., Acc. Chem. Res, 1993, 26, 266), thioamide (Sherman D. B. and Spatola, A. F. J. Am. Chem. Soc., 1990, 112, 433), thioester, phosphonate, ketomethylene (Hoffman, R. V. and Kim, H. O. J. Org. Chem., 1995, 60, 5107), hydroxymethylene, fluorovinyl (Allmendinger, T. et al., Tetrahydron Lett., 1990, 31, 7297), vinyl, methyleneamino (Sasaki, Y and Abe, J. Chem. Pharm. Bull. 1997 45, 13), methylenethio (Spatola, A. F., Methods Neurosci, 1993, 13, 19), alkane (Lavielle, S. et. al., Int. J. Peptide Protein Res., 1993, 42, 270) and sulfonamido (Luisi, G. et al. Tetrahedron Lett. 1993, 34, 2391).

In some embodiments, the compounds comprising a disubstituted β amino acid only contain such disubstituted β amino and one further amino acid, i.e. are compounds having 2 amino acids joined by an amide bond. Such molecules could be considered dipeptides because of the amide bond; however the amide bonds in these compounds are in fact non-scissile due to the disubstitution of the β amino acid and as such these compounds could be considered peptidomimetics. For the purposes of the present disclosure, such compounds (and larger molecules with more amino acids) are considered to be peptides rather than peptidomimetics, due to the presence of an amide bond. This allows for clarity of nomenclature without requiring testing of whether, or to what extent, a given amide bond is scissile. In other words, if all the amino acids in a compound comprising a disubstituted β amino acid as described herein are linked by amide bonds, the compound is considered a peptide, even if one or more of the amide bonds is not readily scissile.

In some embodiments, the comprising a disubstituted β amino is a peptidomimetic which will typically have identifiable sub-units which are approximately equivalent in size and function to amino acids. Peptidomimetics will generally have groups equivalent to the R groups of amino acids and discussion herein of suitable R groups and of N and C terminal modifying groups applies, mutatis mutandis, to peptidomimetic compounds.

As is discussed in the text book referenced above, as well as replacement of amide bonds, peptidomimetics may involve the replacement of larger structural moieties with di- or tripeptidomimetic structures and in this case, mimetic moieties involving the peptide bond, such as azole-derived mimetics may be used as dipeptide replacements. Peptidomimetics and thus peptidomimetic backbones wherein just the amide bonds have been replaced as discussed above are, however, preferred.

Suitable peptidomimetics include reduced peptides where the amide bond has been reduced to a methylene amine by treatment with a reducing agent e.g. borane or a hydride reagent such as lithium aluminium-hydride. Such a reduction has the added advantage of increasing the overall cationicity of the molecule.

Other peptidomimetics include peptoids formed, for example, by the stepwise synthesis of amide-functionalised polyglycines. Some peptidomimetic backbones will be readily available from their peptide precursors, such as peptides which have been permethylated, suitable methods are described by Ostresh, J. M. et al. in Proc. Natl. Acad. Sci. USA (1994) 91, 11138-11142. Strongly basic conditions will favour N-methylation over O-methylation and result in methylation of some or all of the nitrogen atoms in the peptide bonds and the N-terminal nitrogen.

Preferred peptide bond replacements include esters, polyamines and derivatives thereof as well as substituted alkanes and alkenes, particularly aminomethyl and ketomethylene. The peptidomimetics will preferably have N and C termini which may be modified as discussed herein.

In some embodiments, the compound comprising a disubstituted β amino acid is a compound (i.e. peptide, peptidomimetic or amino acid derivative) that incorporates a group of formula (VI):

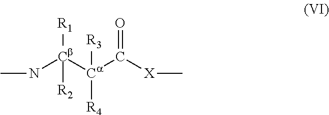

wherein X and N in formula (VI) have their normal valencies and thus will be further substituted as they are bound to other parts of the compound, e.g. further amino acids or N- or C-terminal capping groups and wherein any 2 from R₁, R₂, R₃ and R₄ are hydrogen atoms and 2 are substituting groups, which may be the same or different, comprise at least 7 non-hydrogen atoms, are lipophilic and include a cyclic group, said cyclic group not being attached directly either to the α or β carbon atom but optionally being linked or fused to a cyclic group in the other substituting group, where cyclic groups are fused the combined total number of non-hydrogen atoms for the two substituting groups is at least 12, and wherein X represents O, C, N or S.

It will be appreciated that the minimum figure of 12 for the combined total of non-hydrogen atoms in the two groups of R₁₋₄ when the cyclic groups of each moiety are fused is arrived at by adding the minimum number for the unfused groups (7+7=14) and subtracting 2 because two of the non-hydrogen atoms effectively participate in ring formation in each group. Preferably the combined total of non-hydrogen atoms in the two groups of R₁₋₄ when the cyclic groups of each moiety are fused is 14. Complex fused and linked groups can be envisaged where the two groups attached to the C^{α} or CB may contain more than one pair of fused cyclic groups, with or without additional linking bonds between the substituting groups. Nevertheless, the two substituting groups are preferably not fused or linked as molecules in which these groups have greatest flexibility of movement are preferred.

The nitrogen atom in the group of formula (VI) is preferably not bound to any atom of groups R₁₋₄, except, of course, indirectly through C^{β} or C^{α}. Preferably the 5 atoms in the above backbone (N-C^{β}-C^{α}-C-X) are connected to each other only in a linear, not cyclic, fashion.

The substituting groups of R₁₋₄ are generally lipophilic in nature and preferably carry no charge and preferably have no more than two, more preferably no more than one polar group. One or both of the substituting groups of R₁₋₄ preferably contain at least 8, more preferably at least 9 or 10 non-hydrogen atoms, e.g. 7-13, 7-12, 8-12 or 9-11 non-hydrogen atoms. These two substituting groups are preferably the same, if only for ease of synthesis. Preferably the two substituting groups are R₁ and R₂ or R₃ and R₄, R₃ and R₄ being most preferred.

As stated above, the cyclic groups of R₁₋₄ are not attached directly to either the α or β carbon atom because they are spaced therefrom by a chain of 1 to 4, preferably 1 to 3 atoms; these linking atoms may include nitrogen and/or oxygen but will typically be carbon atoms, preferably the linking atoms are unsubstituted. Preferred spacing moieties are shown in the Examples and form part of the substituting groups of R₁₋₄ as defined herein.

X may be substituted or unsubstituted and is preferably an N atom and preferably substituted. When X is N it may form part of an amide bond with a further amino acid. Alternatively, the N atom may be substituted, for example by an aminoalkyl group, e.g. aminoethyl or aminopropyl or dimethylaminoethyl. In some embodiments, the N atom may form part of a cyclic group such as piperazine, which may itself be substituted by alkyl or aminoalkyl groups.

The compounds incorporating a group of formula (VI) will preferably have a modified C terminus, which is preferably amidated.

Previous passages defining preferred substituting groups of the β amino acid apply, mutatis mutandis, to the two substituting groups of R₁₋₄. The compounds incorporating a group of formula I are a preferred sub-set of those described earlier in this application and so all previous passages defining characteristics of the compounds, for example their length and the other amino acids they contain, apply also to these compounds defined by their incorporation of a group of formula VI, and vice versa. Particularly preferred compounds are 1 to 7 or 8 (e.g. 1 to 5), more preferably 1, 2, 3 or 4 amino acids in length. Peptidomimetic molecules will include the same number of subunits but these subunits will typically be linked by amide bond mimics; preferred linkages are discussed above and include esters and aminomethyl and ketomethylene.

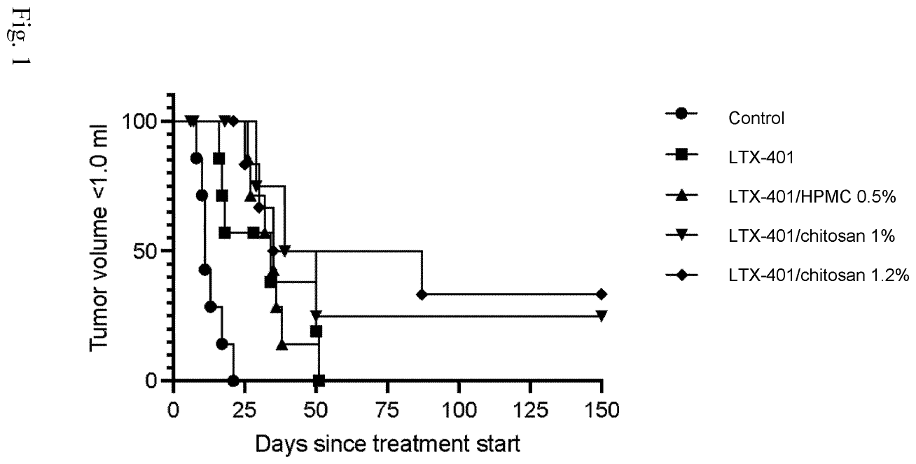

In some embodiments, the compounds comprising a disubstituted β amino acid are the di-peptides (compounds 4a-4n) as shown in FIG. 1 of WO 2011/051692 A1. In some other embodiments, the compounds comprising a disubstituted β amino acid are the amino acid derivatives 1-14 and 23-38 shown in Table 2 and Table 4-7 in Example 2 and FIG. 3 of WO 2011/051692 A1. In yet some other embodiments, the compounds comprising a disubstituted β amino acid are the hepta-peptides 15-22 shown in Table 3 in Example 2 of WO 2011/051692 A1.

The compounds comprising a disubstituted β amino acid may be in salt form, cyclic or esterified, as well as amidated as discussed above.

The compounds comprising a disubstituted β amino acid may be synthesised in any convenient way and such ways are known to the person skilled in the art.

The compounds comprising a disubstituted β amino acid and their synthesis are described in detail in WO 2011/051692 A1, the content of which is incorporated herein by reference.

In a preferred embodiment, the compound comprising a disubstituted β amino acid is LTX-401, i.e. the compound of formula (VII)

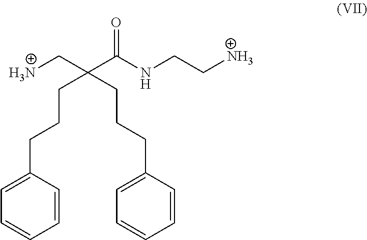 [SMILES: [NH3+]CCNC(=O)C(C[NH3+])(CCCc1ccccc1)CCCc1ccccc1]

preferably in the form of a pharmaceutically acceptable salt, more preferably in the form of its hydrochloride or acetate salt.

### Chitosan and Chitosan Derivatives

Chitosan is a linear polysaccharide composed of randomly distributed β-(1→4)-linked D-glucosamine (deacetylated unit) and N-acetyl-D-glucosamine (acetylated unit).

Chitosan is produced commercially by deacetylation of chitin, which is the structural element in the exoskeleton of crustaceans, such as crabs and shrimp, but can also be manufactured from certain types of fungi and algae. Its main characteristics are the molecular weight and the degree of acetylation (DA) or degree of deacetylation (DDA), which correspond to the molar fractions of deacetylated and acetylated units. Most commercial chitosans have molecular weights ranging from <50-2000 kDa, with an average DDA of 50-100%. Based on molecular weight, chitosan can be grouped into very low molecular weight (typically <50 kDa), low molecular weight (typically 50 to <100 kDa), medium molecular weight (typically 100-1000 kDa), and high molecular weight (typically >1000 kDa). In some embodiments, the chitosan for use in the present disclosure has a DDA of about 50 to 99%, such as 70 to 99%, such as 80%, preferably of about 70% and above, e.g., 75%, 80%, 85%, 86%, 87%, 88%, 89%, 90%, 91%, 92%, 93%, 94%, 95%, 96%, 97%, 98% or 99%.

The chitosan for use in the present disclosure may be a very low molecular weight chitosan, low molecular weight chitosan, medium molecular weight chitosan or high molecular weight chitosan, preferably a very low molecular weight chitosan, low molecular weight chitosan or a medium molecular weight chitosan.

Chitosan contains active functional groups that can undergo chemical reactions and the modification of chitosan by e.g. acylation, carboxylation, alkylation, and quaternization leads to chitosan derivatives with properties different from chitosan, such as improved solubility or bioactivity, but retaining the unique original pharmacological properties of chitosan (see Wang et al. Int. J. Mol. Sci 21 (2), 2020, 487, doi: 10.3390/ijms21020487).

The chitosan derivative for use in the present disclosure may also be a salt of a chitosan derivative, such as a pharmaceutically acceptable salt of chitosan, such as a hydrochloride salt. In the following, the term “chitosan derivative” relates to a chitosan derivative and its pharmaceutically acceptable salts.

Chitosan derivatives for use the present disclosure include O- and N-acylated chitosan, O- and N-alkylated chitosan, chitosan modified by introduction of a hydrophilic group, such as a carboxylic acid group, quaternary ammonium group, sulfonic acid group, phosphoric acid group, amino group, ether bonds composed of an oxygen group, hydroxyl group, carboxylate group, and block polyether group.

In some embodiments, the chitosan derivative for use in the present disclosure is a glycated chitosan, obtained by reaction of free amino groups of chitosan and carbonyl groups of reducing monosaccharides and/or oligosaccharides. Examples of such monosaccharides include naturally occurring D-trioses, D-tetroses, D-pentoses, D-hexoses, D-heptoses, and the like, such as D-glucose, D-galactose, D-fructose, D-mannose, D-allose, D-altrose, D-idose, D-talose, D-fucose, D-arabinose, D-gulose, D-hammelose, D-lyxose, D-ribose, D-rhamnose, D-threose, D-xylose, D-psicose, D-sorbose, D-tagatose, D-glyceraldehyde, dihydroxyacetone, D-erythrose, D-threose, D-erythrulose, D-mannoheptulose, D-sedoheptulose and the like. Examples of such oligosaccharides include fructo-oligosaccharides (FOS), galacto-oligosaccharides (GOS), mannan-oligosaccharides (MOS) and the like. An example of a glycated chitosan is galactochitosan. Glycated chitosan and its synthesis is described for instance in U.S. Pat. No. 5,747,475, WO 2002/040055 A2 and WO 2013/109732 A2, the content of which is incorporated herein by reference.

In some embodiments, the glycated chitosan for use in the present disclosure possesses from about 0.1% to about 90% percent glycation of its otherwise free amino groups, such as from about 0.1% to about 30% or such as from about 2 to 15%, such as about 12.5%. In some embodiments, the glycated chitosan for use in the present disclosure has a molecular weight from about 50 kD to about 2000 kD, such as from about 50 kD to about 1500 kD, such as about 250 kD or 300 kD. In some embodiments, the glycated chitosan for use in the present disclosure has a DDA of about 50 to 99%, such as 70 to 99%, such as 80%. In some embodiments, the glycated chitosan for use in the present disclosure has a DDA of about 50 to 99%, such as 70 to 99%, such as 80%, possesses from about 0.1% to about 90% percent glycation of its otherwise free amino groups, such as from about 0.1% to about 30% or such as from about 2 to 15%, such as about 12.5% and has a molecular weight from about 50 kD to about 2000 kD, such as from about 50 kD to about 1500 kD, such as about 250 kD or about 300 kD.

### Composition Comprising the Oncolytic Peptide

The composition may comprise one oncolytic peptide or several different oncolytic peptides.

The composition comprising the oncolytic peptide is generally a composition comprising the oncolytic peptide and a physiologically acceptable carrier. In some embodiments, said composition is a pharmaceutical composition.

In some embodiments, the physiologically acceptable carrier is water, saline or an aqueous buffer. In some embodiments, the physiologically acceptable carrier is water, e.g. water for injection (WFI). In some other embodiments, the physiologically acceptable carrier is water comprising a salt, e.g. sodium chloride (saline). In yet some other embodiments, the physiologically acceptable carrier is an aqueous buffer or a mixture of several different aqueous buffers. In some embodiments, the composition comprises a mixture of several different physiologically acceptable carriers.

Buffers may be used to maintain the pH of the composition within the desired range. Preferably, the composition has a pH of from 4.0 to 7.4, more preferably of from 5.0 to 7.0, and even more preferably of from 5.0 to 6.5, such as 6.0.

The composition comprising the oncolytic peptide is preferably a solution of the oncolytic peptide in the physiologically acceptable carrier, more preferably a solution of the oncolytic peptide in water, saline or an aqueous buffer or a mixture of several different aqueous buffers.

In some embodiments, the composition comprises the oncolytic peptide at a concentration of from 0.1 to 30 mg/ml, such as 1 to 5 mg/ml or 1 to 30 mg/ml, preferably 4 to 25 mg/ml, or more preferably 6 to 20 mg/ml. The aforementioned amounts of peptide refer, where applicable (i.e. where the oncolytic peptide actually is a peptide) to the net peptide amount and for oncolytic peptides as defined herein which are in the form of their salts, to the free base.

The net peptide amount is the gross peptide obtained after its synthesis corrected for its purity (as determined by analysis, e.g. determined by HPLC) and the net peptide content (as determined by analysis, e.g. determined by elemental analysis). The net peptide content is the fraction of the peptide in question relative to counter-ions, residual water and peptidic impurities. The net peptide (in %) is calculated as follows:

$$\frac{Net peptide content ( \% ) \times purity ( \% )}{100 \%}$$

The result gives the percentage of net peptide in the gross peptide.

In some embodiments, the composition comprises one or more further pharmaceutically acceptable excipients, such as those excipients well known in the art that are used in pharmaceutical compositions/medical products comprising aqueous solutions of active ingredients.

In some embodiments, the composition comprising the oncolytic peptide may be a solution contained in a container, e.g. sealed container, ready for use. In other embodiments, the composition may be prepared just prior to it being used/administrated, e.g. by reconstituting, e.g. dissolving, the oncolytic peptide which is provided in solid form (e.g. in lyophilized form) in the pharmaceutically acceptable carrier.

In some embodiments, the composition comprises LTX-401 and a pharmaceutically acceptable carrier. In some embodiments, the composition is a solution of LTX-401 in the pharmaceutically acceptable carrier which is contained in a sealed container ready for use.

### Composition Comprising a 9-Mer Peptide

For a composition comprising one or more 9-mer peptides, such composition comprises, in some embodiments, one or more of the following aqueous buffers: acetic acid buffer, acetate buffer, aspartic acid buffer, sodium benzoate buffer, benzoic acid buffer, carbonic acid buffer, citric acid buffer, glucono-delta-lactone buffer, glycine buffer, glycine HCl buffer, histidine buffer, histidine HCl buffer, hydrobromic acid buffer, phosphate buffer (such as PBS), sodium succinate buffer, disodium succinate buffer, succinic acid buffer, sulfuric acid buffer, tartaric acid buffer and/or sodium tartrate buffer. Preferably, the aqueous buffer is an acetic acid/acetate buffer, citric acid buffer and/or a phosphate buffer.

For example, in some embodiments, the composition comprising a 9-mer peptide comprises one or more preservative agents. The table below lists examples of preferred preservative agents. Also listed are suitable concentration ranges for these agents if present in composition:

| Preservative agent | Concentration (w/w) |
|---|---|
| Benzalkonium chloride | 0.004% to 0.02% |
| Benzyl alcohol | 0.75 to 5% |
| Chlorobutanol | 0.25 to 0.5% |
| Metacresol (m-cresol) | 0.1 to 0.35% |
| Methylparaben | 0.01% to 0.2% |
| Phenol | 0.15 to 0.5% |
| Potassium sorbate | 0.05% to 0.2% |
| Propylparaben | 0.005 to 0.035 |
| Thimerosal | 0.001% to 0.012% |

Benzyl alcohol is a particularly preferred preservative agent, preferably this is present at 0.75% to 2% w/w, preferably 1% w/w. Another preferred preservative agent is phenol, which is preferably present at 0.15% to 0.5%.

In some embodiments, the composition comprising a 9-mer peptide comprises one or more chelating agents to chelate metal ions. The table below lists examples of preferred chelating agents. Also listed are suitable concentration ranges for these agents if present in the composition:

| Chelating Agents | Concentration (w/w) |
|---|---|
| Calcium disodium EDTA | 0.01 to 0.1% |
| Calcium versetamide sodium | 2 to 3.5% |
| Calteridol | 0.01 to 0.03% |
| Disodium EDTA | 0.01 to 0.11% |
| DTPA | 0.04 to 1.2% |
| Sodium EDTA | 0.1 to 0.3% |

EDTA = ethylenediaminetetraacetic acid,
DTPA = diethylenetriamine pentaacetate

Preferably the chelating agent is disodium EDTA and/or sodium EDTA. Preferably the chelating agent, such as disodium or sodium EDTA, is present at about 0.1% w/w.

In some embodiments, the composition comprising a 9-mer peptide comprises one or more antioxidants. The table below lists examples of preferred antioxidants. Also listed are suitable concentration ranges for the antioxidants if present in the composition:

| Antioxidants | Concentration (w/w) |
|---|---|
| Ascorbic Acid | 0.02 to 0.15% |
| Ascorbyl palmitate | 0.01 to 0.03% |
| Citric acid | 0.10 to 2.0% |
| Erythorbic acid | 0.02 to 0.1% |
| Methionine | 0.01 to 0.15% |
| Monothioglycerol | 0.1 to 1% |
| Potassium metabisulfite | 0.05% to 0.10% |
| Sodium ascorbate | 0.1 to 4.8% |
| Sodium bisulfite | 0.1 to 0.15% |
| Sodium formaldehyde sulfoxylate | 0.1 to 0.15% |
| Sodium thiosulfate | 0.1 to 0.2% |
| Thiourea | 0.002 to 0.008% |

A preferred antioxidant is thiourea.

In some embodiments, the composition comprising a 9-mer peptide comprises one or more tonicity modifiers, such as dextrose, glycerine, lactose, mannitol, potassium chloride, sodium chloride and/or sorbitol, to obtain an isotonic composition which reduces pain experienced by a patient to whom the composition is administered. Preferably, the tonicity modifier is sodium chloride.

In some embodiments, the composition comprising a 9-mer peptide comprises one or more stabilizers which inhibit aggregation of the peptide. Such stabilizers include anti-stacking agents. Examples of suitable stabilizers are dimethyl sulfoxide, dimethylacetamide, ethanol, glycerol, mannitol, N-methyl-2-pyrrolidone, polyethylene glycol (PEG) 200, PEG 300, PEG 350, PEG 400, PEG 600, propylene glycol and sorbitol. PEG stabilizers, e.g. PEG 200, are particularly preferred. Such stabilizers, e.g. PEG stabilizers, may be present at 1 to 8% w/w, preferably 2 to 7% w/w, e.g. 3 to 6% w/w.

In some embodiments, the composition comprising a 9-mer peptide comprises one or more viscosity enhancers, such as acacia gum, agar, cellulose derivatives, such as methylcellulose, hydroxypropyl cellulose, hydroxypropyl methylcellulose (HPMC), hydroxyethyl cellulose, carboxymethylcellulose calcium, carboxymethylcellulose sodium, gelatine, hyaluronic acid, maltodextrin, pectin, poloxamer 407, PEG, polyethylene oxide, povidone (PVP), starches like corn starch and tapioca starch, tragacanth and/or β-cyclodextrins.

Enhanced viscosity (compared to that of water) of the composition comprising a 9-mer peptide can effectively slow the dispersion of the peptide from the site of administration. If the composition is administered intratumorally, enhanced viscosity can extend the time the composition stays within the tumor and does not leak out, slowing dispersion of the peptide by the blood and prolonging the time the peptide is in direct contact with the tumor cells. In this way, the efficacy of the peptide may be increased and/or the toxicity reduced.

Thus, in some embodiments, the composition comprising a 9-mer peptide comprises one or more viscosity enhancers and has a viscosity suitable for injection, e.g. injection into a tumor. In some embodiments, the composition comprising the oncolytic peptide has a viscosity of from 1.5, 2, 2.5 or 3 cP to 300 cP, e.g. from 2.5 to 250 cP, such as 5 to 100 cP or 10 to 200 cP and 20 to 240 cP, preferably of from 40 to 180 cP, e.g. from 45 to 150 cP or from 30 to 100 cP and more preferably 1.5 to 10 cP, such as 2, 3, 4, 5, 6, 7, 8, 9 or 10 cP or of from 10 to 15 cP or 15 to 20 cP or 20 to 35 cP or 35 to 50 cP or 50 to 100 cP.

Viscosity enhancers are preferably present at 0.4 to 1.2% w/w, e.g. 0.5 to 1% w/w.

Preferably, the one or more viscosity enhancers are selected from the group consisting of hydroxyethyl cellulose, hydroxypropyl cellulose, HPMC, poloxamer, PEG, and PVP.

If the composition comprising a 9-mer peptide is prepared prior to it being administered, aforementioned excipients like preservative agents, antioxidants or stabilizing agents may not be needed or, if present in the reconstituted composition, are preferably present in the pharmaceutically acceptable carrier. In some embodiments, if the composition is prepared prior to it being used/administered, any of the aforementioned excipients are present in the pharmaceutically acceptable carrier.

In some embodiments, the composition comprises LTX-315 and a pharmaceutically acceptable carrier, preferably LTX-315 having the amino acid sequence of SEQ ID NO: 23, or a pharmaceutically acceptable salt thereof, preferably the acetate salt thereof.

In some embodiments, such composition is a solution of LTX-315 in the pharmaceutically acceptable carrier which is contained in a sealed container ready for use. In some other embodiments, such composition is a solution of LTX-315 which is reconstituted prior to use/administration by dissolving such LTX-315 in solid form (e.g. as a lyophilized powder) in a pharmaceutically acceptable carrier.

### Manufacture of a Composition Comprising the Oncolytic Peptide

In some embodiments, the composition comprising the oncolytic peptide is a pharmaceutical grade product. In some embodiments, the composition comprising the oncolytic peptide is a sterile composition. In some embodiments, the composition comprising the oncolytic peptide is a pharmaceutical composition.

The composition comprising the oncolytic peptide may be manufactured using any conventional method.

In some embodiments, the composition comprising the oncolytic peptide is a solution contained in a sealed container ready for use. To manufacture such product, the oncolytic peptide in its solid form, e.g. in the form of a (lyophilized) powder, may be dissolved in the pharmaceutically acceptable carrier under aseptic manufacturing conditions, the resultant composition is preferably sterile filtered and then filled into a container and sealed. Alternatively, the oncolytic peptide may be dissolved in the pharmaceutically acceptable carrier, the resultant composition is then filled into a container, sealed and the container is then autoclaved to obtain a sterile composition. If excipients are present in the composition, these are either mixed or dissolved in the pharmaceutically acceptable carrier or mixed or dissolved in the solution comprising the oncolytic peptide, depending on the nature and amount of the excipient.

In some other embodiments, the composition comprising the oncolytic peptide is prepared just prior to it being used/administrated, e.g. by reconstituting, e.g. dissolving, the oncolytic peptide which is provided in solid form (e.g. in the form of a lyophilized powder) in the sterile pharmaceutically acceptable carrier. To manufacture such product, in some embodiments, the oncolytic peptide is dissolved, sterile-filtered and lyophilized. In some other embodiments, the oncolytic peptide is dissolved, lyophilized and the lyophilized oncolytic peptide is sterilized by gamma irradiation.

Composition Comprising Chitosan and/or a Chitosan Derivative

The composition may comprise one or more chitosans, e.g. several different chitosans, e.g. chitosans of different molecular weight and/or different DDA and/or may comprise one or more chitosan derivatives, e.g. several different chitosan derivatives, e.g. comprising the same modification but different degree of modification or comprising different modifications.

The composition comprising chitosan and/or a chitosan derivative is generally a composition comprising chitosan and/or a chitosan derivative and a physiologically acceptable carrier. In some embodiments, the composition comprising chitosan and/or a chitosan derivative is a pharmaceutical composition.

In some embodiments, the composition comprising chitosan and/or a chitosan derivative is a dispersion, suspension or a solution of chitosan and/or a chitosan derivative in the physiologically acceptable carrier.

In some embodiments, the physiologically acceptable carrier is water, saline or an aqueous buffer. In some embodiments, the physiologically acceptable carrier is water, e.g. water for injection (WFI). In some other embodiments, the physiologically acceptable carrier is water comprising a salt, e.g. sodium chloride (saline) or other salts such as potassium chloride, calcium chloride or magnesium chloride or an aqueous buffer comprising a salt, e.g. sodium chloride or other salts such as potassium chloride, calcium chloride or magnesium chloride. In some embodiments, the composition comprises a mixture of several different physiologically acceptable carriers. In yet some other embodiments, the physiologically acceptable carrier is an aqueous buffer or a mixture of several different aqueous buffers.

Buffers may be used to maintain the pH of the composition within the desired range. Typically, the composition has a pH of from 4.0 to 7.5. Preferably, the composition has a pH of from 5.0 to 7.4, more preferably of from 5.0 to 7.0, such as 6.5 or 6.0.

In some embodiments, the composition comprising chitosan and/or a chitosan derivative has a viscosity suitable for injection, e.g. suitable for injection into a tumor of a subject. In some embodiments, the composition comprising chitosan and/or a chitosan derivative has a viscosity of from 1.0, 1.5, 2, 2.5 or 3 cP to 300 cP, e.g. from 2.5 to 250 cP, such as 5 to 100 cP or 10 to 200 cP and 20 to 240 cP, preferably of from 40 to 180 cP, e.g. from 45 to 150 cP or from 30 to 100 cP and more preferably 1.5 to 10 cP, such as 2, 3, 4, 5, 6, 7, 8, 9 or 10 cP or of from 10 to 15 cP or 15 to 20 cP or 20 to 35 cP or 35 to 50 cP or 50 to 100 cP In some embodiments, the composition comprises glycated chitosan and has a viscosity of from 1 to 100 cP.

In some embodiments, the composition comprises 0.1 to 5% w/w of the chitosan and/or the chitosan derivative, such as 0.1 to 2.5% w/w such as 0.5%, 1%, 1.5% or 2% w/w.

In some the composition comprises one or more further pharmaceutically acceptable excipients, such as those excipients well known in the art that are used in medical products comprising aqueous dispersions, suspensions or solutions of active ingredients.

In some embodiments, the composition comprises glycated chitosan. In some other embodiments, the composition comprises galactochitosan.

Manufacture of a Composition Comprising Chitosan and/or a Chitosan Derivative

In some embodiments, the composition comprising chitosan and/or a chitosan derivative is a pharmaceutical grade product. In some embodiments, the composition comprising chitosan and/or a chitosan derivative is a sterile composition. In some embodiments, the composition comprising chitosan and/or a chitosan derivative is a pharmaceutical composition.

The composition comprising chitosan and/or a chitosan derivative may be manufactured using any conventional method. In some embodiments, chitosan and/or a chitosan derivative is dispersed, suspended or dissolved in the physiologically acceptable carrier and the resulting composition is sterile filtered and then filled into a container and sealed. In some embodiments, chitosan and/or a chitosan derivative is dispersed, suspended or dissolved in the physiologically acceptable carrier and the resulting composition is autoclaved. In some embodiments, the composition comprising chitosan and/or a chitosan derivative is prepared just prior to it being used/administered, e.g. by reconstituting, e.g. dissolving, dispersing or suspending, chitosan and/or a chitosan derivative which is provided in solid form (e.g. in lyophilized form) in the sterile pharmaceutically acceptable carrier. To manufacture such product, in some embodiments, chitosan and/or a chitosan derivative is dissolved, sterile-filtered and lyophilized. In some other embodiments, chitosan and/or a chitosan derivative is dissolved, lyophilized and the lyophilized compound is sterilized by gamma irradiation.

### Kit for Compositions (a) and (b)

The compositions (a) and (b) may be provided in the form of a kit comprising both such compositions or comprising the parts/components to prepare compositions (a) and (b). In some embodiments, the kit comprises the compositions (a) and (b), each in a (sealed) container.

The kit will typically comprise instructions for use of compositions (a) and (b).

In some other embodiments, the kit comprises the oncolytic peptide and a physiologically acceptable carrier to prepare composition (a) and the chitosan and/or a chitosan derivative and a physiologically acceptable carrier to prepare composition (b). In some embodiments, the physiologically acceptable carrier for preparing composition (a) is the same as the physiologically acceptable carrier for preparing composition (b). In such embodiments, the kit may comprise 3 containers or 3 compartments comprised in 1 or 2 containers, one comprising the oncolytic peptide, one comprising the chitosan and/or a chitosan derivative (e.g. both in solid form) and one comprising the physiologically acceptable carrier for preparing composition (a) and (b). In some other embodiments, the physiologically acceptable carriers for preparing composition (a) and (b) are different. In such embodiments, the kit may comprise 4 containers or 4 compartments comprised in 1, 2 or 3 containers, one comprising the oncolytic peptide, one comprising the chitosan and/or a chitosan derivative (e.g. both in solid form), one comprising the physiologically acceptable carrier for preparing composition (a) and one comprising the physiologically acceptable carrier for preparing composition (b).

The kit will typically comprise instructions for preparing compositions (a) and (b), e.g. reconstituting the oncolytic peptide and the chitosan and/or chitosan derivative in the physiologically acceptable carrier prior to use and instructions for use of compositions (a) and (b).

In yet some other embodiments, the kit comprises composition (a), the chitosan and/or a chitosan derivative (e.g. in solid form) and a physiologically acceptable carrier to prepare composition (b).

The kit will typically comprise instructions for preparing composition (b), e.g. reconstituting the chitosan and/or chitosan derivative in the physiologically acceptable carrier prior to use and instructions for use of compositions (a) and (b).

In yet some other embodiments, the kit comprises composition (b), the oncolytic peptide (e.g. in solid form) and a physiologically acceptable carrier to prepare composition (a).

The kit will typically comprise instructions for preparing compositions (a), e.g. reconstituting the oncolytic peptide in the physiologically acceptable carrier prior to use and instructions for use of compositions (a) and (b).

Composition Comprising i) an Oncolytic Peptide and ii) Chitosan and/or a Chitosan Derivative

A composition comprising i) an oncolytic peptide and ii) chitosan and/or a chitosan derivative may also be denoted herein a “combined composition”.

The combined composition may comprise one oncolytic peptide or several different oncolytic peptides. Further, the combined composition may comprise one or more chitosans, e.g. several different chitosans, e.g. chitosans of different molecular weight and/or different DDA and/or may comprise one or more chitosan derivatives, e.g. several different chitosan derivatives, e.g. comprising the same modification but different degree of modification or comprising different modifications.

The combined composition is generally a composition comprising i) and ii) and a physiologically acceptable carrier. In some embodiments, the combined composition is a pharmaceutical composition.

In some embodiments, the combined composition is a dispersion, suspension or a solution, i.e. dispersion, suspension or solution of i) and ii) in the physiologically acceptable carrier. In some embodiments, the oncolytic peptide is dissolved in the physiologically acceptable carrier while chitosan and/or a chitosan derivative is dispersed or suspended in the dissolved oncolytic peptide.

In some embodiments, the physiologically acceptable carrier is water, saline or an aqueous buffer. In some embodiments, the physiologically acceptable carrier is water, e.g. water for injection (WFI). In some other embodiments, the physiologically acceptable carrier is water comprising a salt, e.g. sodium chloride (saline) or other salts such as potassium chloride, calcium chloride or magnesium chloride or an aqueous buffer comprising a salt, e.g. sodium chloride or other salts such as potassium chloride, calcium chloride or magnesium chloride. In yet some other embodiments, the physiologically acceptable carrier is an aqueous buffer or a mixture of several different aqueous buffers.

Buffers may be used to maintain the pH of the combined composition within the desired range. Preferably, the combined composition has a pH of from 4.0 to 7.4, more preferably of from 5.0 to 7.0, such as 6.0 or 6.5.

The combined composition comprises the oncolytic peptide at a concentration of from 0.1 to 30 mg/ml, such as 1 to 5 mg/ml or 1 to 30 mg/ml, preferably 4 to 25 mg/ml, or more preferably 6 to 20 mg/ml. The aforementioned amounts of peptide refer, where applicable (i.e. where the oncolytic peptide is actually a peptide) to the net peptide amount and to the free base.

The combined composition comprises 0.1 to 5% w/w of the chitosan and/or the chitosan derivative, such as 0.1 to 2.5% w/w such as 0.5% to 1.5%, such as 0.6% to 1% or 1% to 1.5% or 1.5% to 2% or 2% to 2.5% w/w.

In some embodiments, the combined composition has a viscosity suitable for injection, e.g. for injection into a tumor of a subject. In some embodiments, the combined composition has a viscosity of from 1.0, 1.5, 2, 2.5 or 3 cP to 300 cP, e.g. from 2.5 to 250 cP, such as 5 to 100 cP or 10 to 200 cP and 20 to 240 cP, preferably of from 40 to 180 cP, e.g. from 45 to 150 cP or from 30 to 100 cP and more preferably 1.5 to 10 cP, such as 2, 3, 4, 5, 6, 7, 8, 9 or 10 cP or of from 10 to 15 cP or 15 to 20 cP or 20 to 35 cP or 35 to 50 cP or 50 to 100 cP.

For a combined composition comprising a 9-mer peptide, such composition may further comprise the excipients described in the respective section herein, i.e. the section “Composition comprising a 9-mer peptide”.

In some embodiments, the combined composition comprises LTX-401. In some other embodiments, the combined composition comprises LTX-315. In yet some other embodiments, the combined composition comprises glycated chitosan, such as galactochitosan. In yet some other embodiments, the combined composition comprises LTX-401 and glycated chitosan, such as galactochitosan or comprises LTX-315 and glycated chitosan, such as galactochitosan.

### Manufacture of a Combined Composition

In some embodiments, the combined composition is a pharmaceutical grade product. In some embodiments, the combined composition is a sterile composition. In some embodiments, the combined composition is a pharmaceutical composition.

The combined composition may be manufactured using any conventional method. In some embodiments, chitosan and/or a chitosan derivative is dispersed, suspended or dissolved in the physiologically acceptable carrier and the oncolytic peptide is dissolved in said dispersion, suspension or solution. In some other embodiments, chitosan and/or a chitosan derivative is dispersed, suspended or dissolved in a solution of the oncolytic peptide in the physiologically acceptable carrier. The solution of the oncolytic peptide may be obtained by dissolving the oncolytic peptide in its solid form, e.g. in the form of a powder in the physiologically acceptable carrier. In some embodiments, the combined composition is sterile filtered and then filled into a container and sealed or is filled into a container and autoclaved.

### Kit for the Combined Composition

The parts/components to prepare the combined may be provided in a kit, i.e. the combined composition may be prepared before use/administration.

In some embodiments, the kit comprises compositions (a) and (b) as described herein and instructions to prepare the combined composition. The kit will typically comprise instructions for use of the combined composition.

In some other embodiments, the kit comprises the oncolytic peptide, the chitosan and/or a chitosan derivative (e.g. both in solid form) and a physiologically acceptable carrier to prepare the combined composition. In such embodiments, the kit may comprise 3 containers or 3 compartments comprised in 1 or 2 containers, one comprising the oncolytic peptide, one comprising the chitosan and/or a chitosan derivative (e.g. both in solid form) and one comprising the physiologically acceptable carrier for preparing the combined composition or may comprise 2 containers or 2 compartments comprised in 1 container, one comprising a mixture of the oncolytic peptide and the chitosan and/or a chitosan derivative and one comprising the physiologically acceptable carrier for preparing the combined composition.

The kit will typically comprise instructions for preparing the combined composition, e.g. reconstituting the oncolytic peptide and the chitosan and/or chitosan derivative or the mixture thereof in the physiologically acceptable carrier prior to use and instructions for use of the combined composition.

In yet some other embodiments, the kit comprises composition (a) and the chitosan and/or a chitosan derivative (e.g. in solid form).

The kit will typically comprise instructions for preparing the combined composition, e.g. reconstituting the chitosan and/or chitosan derivative in composition (a) prior to use and instructions for use of the combined composition.

In yet some other embodiments, the kit comprises composition (b) and the oncolytic peptide (e.g. in solid form).

The kit will typically comprise instructions for preparing the combined composition, e.g. reconstituting the oncolytic peptide in composition (b) prior to use and instructions for use of the combined composition.

### Method of Treating a Subject Having a Tumor/Use of the Compositions as Disclosed Herein in a Method of Treating a Subject Having a Tumor

The compositions (a) and (b) disclosed herein are used concurrently, sequentially or as a combined composition (i.e. a combined composition as disclosed herein) in a method of treating a subject having a tumor, e.g. are administered to said subject concurrently, sequentially or as a combined composition.

In some embodiments, compositions (a) and (b) are administered concurrently, i.e. at the same time. In this context, concurrently means simultaneously (e.g. by the same or different way of administration) or within a short period of time, e.g. within minutes or hours or longer, but on the same day. In some embodiments, compositions (a) and (b) are administered concurrently, with the composition (b) comprising chitosan and/or a chitosan derivative being administered first, followed by administration of the composition (a) comprising the oncolytic peptide. In some other embodiments, compositions (a) and (b) are administered concurrently, with the composition (a) comprising the oncolytic peptide being administered first, followed by administration of the composition (b) comprising chitosan and/or a chitosan derivative.

In some other embodiments, compositions (a) and (b) are administered sequentially. In this context, sequentially means on different days, e.g. with a day/days or a week/weeks apart, e.g. by the same or different way of administration. In some embodiments, compositions (a) and (b) are administered sequentially, with the composition (b) comprising chitosan and/or a chitosan derivative being administered first, followed by administration of the composition (a) comprising the oncolytic peptide. In some other embodiments, compositions (a) and (b) are administered sequentially, with the composition (a) comprising the oncolytic peptide being administered first, followed by administration of the composition (b) comprising chitosan and/or a chitosan derivative.

In some embodiments, compositions (a) and (b) are administered each only once, i.e. one dose is administered at one point in time in the course of the treatment. In some other embodiments, compositions (a) and (b) are each administered repeatedly, i.e. several doses are administered at several points in time in the course of the treatment. In some other embodiments, one of the compositions (a) and (b) is administered repeatedly while the other is only administered once.

In some embodiments, the composition is the combined composition. In some embodiments, such combined composition is administered only once. In some other embodiments, such combined composition is administered repeatedly in the course of the treatment.

The combined composition and the composition (a) are preferably administered into the tumor. In some embodiments, said compositions are injected into the tumor. In some other embodiments, said compositions are administered into the tumor by perfusion/infusion of a preferably isolated (including partial isolation) limb, body region or organ which contains said tumor. Any one way of administration or a combination of both ways (injection and perfusion/infusion), of localizing said compositions in the tumor site is acceptable so long as the delivery mechanism ensures sufficient concentration of the components of said compositions in the tumor.

The composition (b) is preferably administered into the tumor and/or the tissue immediately surrounding the tumor. Composition (b) may be administered by injection, perfusion/infusion or a combination of both ways as described above for composition (a) and the combined composition.

In the method of treatment/use of the compositions disclosed herein for treatment, the compositions are preferably administered in a therapeutically effective amount. Such an amount may be administered in one administration, i.e. one dose, or in several administrations, i.e. repetitive doses, i.e. in a series of doses, e.g. over the course of several days, weeks or months.

The actual dose to be administered may vary and depend on the age, weight, gender, medical history, pre-existing conditions and general condition of the subject, the severity of the disease being treated and the judgment of the health care professionals.

The method of treatment/use of the composition disclosed herein for treatment can be continued for as long as the clinician overseeing the patient's care deems the method to be effective and the treatment to be needed.

In some embodiments, the method of treatment/use of the composition disclosed herein for treatment is for treating a tumor selected from the group consisting of tumors of breast cancer, ovarian cancer, colon cancer, prostate cancer, bone cancer, colorectal cancer, gastric cancer, skin cancer, small cell lung cancer, non-small cell lung cancer, pancreatic cancer, thyroid cancers, kidney cancer, liver cancer, cancer of the bile duct, brain cancer, cervical cancer, bladder cancer, esophageal cancer, Hodgkin's disease and adrenocortical cancer. In some embodiments, the tumor is a carcinoma, adenocarcinoma, lymphoma, melanoma, blastoma, leukemia, sarcoma and germ cell tumor.

### EXAMPLES

The foregoing written description is considered to be sufficient to enable one skilled in the art to practice the invention. The following Examples are offered for illustrative purposes only and are not intended to limit the scope of the present invention in any way. Indeed, various modifications of the invention in addition to those shown and described herein will become apparent to those skilled in the art from the foregoing description and fall within the scope of the appended claims.

### Example 1: Preparation of Compositions as Disclosed Herein

The compositions below were prepared as follows:

Approximately half of the amount of aqueous acetic acid/acetate buffer or saline was added to a reaction vessel equipped with magnetic stirring means and stirred. LTX-401 in the form of its acetate salt (prepared as described in WO 2011/051692 A1) as a dry powder was added and stirred until dissolved. If present in the composition, chitosan or HPMC was added while stirring. Subsequently, the rest of the buffer or saline was added and the mixture was stirred vigorously until complete dissolution of the polymer. All compositions were autoclaved at 121° C. for 15 minutes.

| | LTX- | CS | CS | HPMC | HPMC | | | |
|---|---|---|---|---|---|---|---|---|
| | 401* | LMW | MMW | LMW | HMW | | | Viscosity |
| | [mg/ml] | [% w/w] | [% w/w] | [% w/w] | [% w/w] | Carrier | pH | [cP] |
| 1 | 24 | | | 1 | | buffer | 5.8 | 371.3 |
| 2 | 24 | | | | 1 | buffer | 5.8 | 2252.6 |
| 3 | 24 | | | 0.5 | | buffer | 5.8 | 40.9 |
| 4 | 24 | | | | 0.5 | buffer | 5.8 | 194.8 |
| 5 | 24 | 1 | | | | buffer | 5.3 | 35.5 |
| 6 | 24 | | 1 | | | buffer | 5.3 | 49.1 |
| 7 | 24 | 1.5 | | | | buffer | 5.2 | 107.1 |
| 8 | 24 | | 1.5 | | | buffer | 5.2 | 175.2 |
| 9 | 24 | | | | | saline | | ntd |
| 10 | 12 | | | | 0.5 | buffer | 5.5 | 238.8 |
| 11 | 12 | 1 | | | | buffer | 5.3 | 35.5 |
| 12 | 12 | | 1 | | | buffer | 5.3 | 52.0 |
| 13 | 12 | | 1.2 | | | buffer | 5.5 | 74.3 |
| 14 | 12 | | 1.3 | | | buffer | 5.3 | 94.4 |
| 15 | 12 | | | | | saline | | ntd |

*24 mg/ml corresponds to 20 mg/ml free base and 12 mg/ml corresponds to 10 mg/ml free base.
ntd: not determined
CS LMW = chitosan low molecular weight, 340 kDa, DAA 85.4% (manufacturer Chitinor, Norway)
CS MMW = chitosan medium molecular weight, 350-600 kDa, DAA 88% (manufacturer Chitinor, Norway)
HPMC LMW = hydroxyproplyl methylcellulose low molecular weight, pharma grade
HPMC metolose 60 SH (LMW) (Safic Alcan, Paris La Dèfense Cedex, France)
HPMC HMW = hydroxyproplyl methylcellulose high molecular weight, pharma grade
HPMC metolose 60 SH (HMW) (Safic Alcan, Paris La Dèfense Cedex, France)

Viscosity was determined by viscometer from Fungilab/Evo expert with LCP spindle. Measurements were done at 25° C. on a sample volume of 20 mL with a speed of 10 rpm and a shear rate of 12.2 s⁻¹ and pH was determined according to Ph. Eur.

### Example 2: In Vitro Cytotoxic Activity

The compositions 1-15 in the table above were tested for their cytotoxic (oncolytic) activity in vitro in 3 cell lines by determining their respective IC50s (half maximal inhibitory concentration), i.e. the concentration necessary to kill 50% of the cells. Cell viability was determined by WST-1 assay (Roche). RPMI 1640 medium without FCS (assay medium) and 1% v/v Triton X-100 in assay medium were used as the negative and positive control, respectively. Three independent experiments/cell line were performed. Stock solutions of compositions 1-15 were prepared comprising 0.8 mg/ml LTX-401 and were diluted in assay medium to 2-200 μg/ml

The 3 cell lines used in this Example were the following (all obtained from ATCC, USA):
143B, a human osteosarcoma cell line
K7M2, a mouse osteosarcoma cell line.
B16F10, a mouse melanoma cell line.

Cells were plated in 96-well flat bottom microtitration plates in aliquots of 100 μl per well and incubated for 16 hours at 37° C. in RPMI plus FCS. The cells were washed once with RPMI without FCS, and 100 μl/well of the diluted compositions 1-15, negative control or positive control was added. The plates were incubated for 4 hours at 37° C. and after that period, 10 μl WST-1 solution was added to each well. The plates were incubated for 2 more hours. 70 μl of the solution in each well was removed and 100 μl acidic isopropanol was added. Absorbance (OD) was measured in each well at 590 nm using a Varioskan™ LUX multimode microplate reader (Thermo Scientific). The cell viability of compositions 1-8 and 10-14 without LTX-401 was determined by WST-1 assay to assess the effect of HPMC or chitosan alone on the cell viability. None of these compositions affected the cell viability.

Based on their IC50 in the 3 different cell lines, their concentration of LTX-401 and their viscosity, compositions 10, 12, 13 and 15 were selected for use in Example 3.

### Example 3: In Vivo Cytotoxic Activity Against Osteosarcoma

The following compositions 10, 12, 13 and 15 were selected to be tested in vivo in a syngeneic osteosarcoma mouse model by studying the effect of administration of the composition into the tumor:
10: LTX-401, 10 mg/ml (free base), 0.5% w/w HPMC HMW
12: LTX-401, 10 mg/ml (free base), 1% w/w chitosan MMW
13: LTX-401, 10 mg/ml (free base), 1.2% w/w chitosan MMW
15: LTX-401, 10 mg/ml (free base)

### Control: Saline

Balb/c mice (Taconic Biosciences) were inoculated with K7M2 tumor cells on day 0 by subcutaneous injection of 8×10⁶ tumor cells in the right rear flank. The mice were divided in 5 groups of 7 mice each, and treatment was initiated when the tumors had reached the desired size of approximately 5 mm in diameter. The respective compositions were administered by injection into the tumor (50 μl) on 3 consecutive days. The mice were monitored during the study by measuring the tumors and weighing the animals regularly. The mice were followed until the maximum tumor burden of 1.5 ml volume or 20 mm length was reached, or until serious adverse events occurred (i.e. wound formation or ulceration at tumor/injection site), then the mice were sacrificed. A calliper was used for tumor size measurements and weighing and physical examination were used as health control.

The results are shown in FIG. 1: Tumor growth in mice with a LTX-401 composition was slower compared to tumor growth in mice that received saline alone. Essentially no difference in tumor growth was observed between the group of mice that were treated with LTX-401 alone and the group of mice that were treated with LTX-401/0.5% HPMC. Mice that were treated with a composition comprising LTX-401 and chitosan showed a markedly slower tumor growth and even after 150 days, 1 mouse in the group that received LTX-401/1% chitosan and 2 mice in the group that received LTX-401/1.2% chitosan were still alive and in remission (no visible or palpable tumor).

### Example 4: In Vivo Cytotoxic Activity Against Melanoma

The following compositions were prepared as described in Example 1 in aqueous acetic acid/acetate buffer, pH 5.2. The following chitosan was used in this study: chitosan medium molecular weight, 330 kDa, DAA 86% (manufacturer Chitinor, Norway).
1: LTX-401, 5 mg/ml (free base)
2: Chitosan, 1.2% (w/w)
3: LTX-401, 5 mg/ml (free base) and chitosan, 1.2% (w/w)
4: Control: aqueous acetic acid/acetate buffer

Immunocompetent C57BL/6NRj mice (JanVier Labs, France) were inoculated with B16F1 melanoma cells on day 0 by subcutaneous injection of 5×10⁴ tumor cells per mouse/50 μl RPMI-1640 in the skin on the abdomen of the animal. The mice were divided into 4 groups of 10 mice each, and treatment was initiated when the tumors were palpable and reached the desired size of approximately 5 mm in diameter (40-80 mm³). The respective compositions 1~4 were administered by injection into the tumor (50 μl) on two consecutive days (day 11 and 12 or day 12 and 13 or day 13 and 14, depending on tumor growth). The mice were monitored during the study by measuring the tumors and weighing the animals regularly. The tumor volume was measured using an electronic caliper and expressed as volume V=(L×W×W)/2, where W is the tumor width and L is the tumor length. The mice were followed until the maximum tumor burden of approximately 12-13 mm in diameter (860-1100 mm³) was reached, metastasis occurred, or until serious adverse events occurred (i.e. wound formation or ulceration at tumor/injection site). If any of these endpoints were reached the mice were sacrificed. A score sheet was used for evaluating the overall health of the animals, based on parameters such as body weight and physical examination.

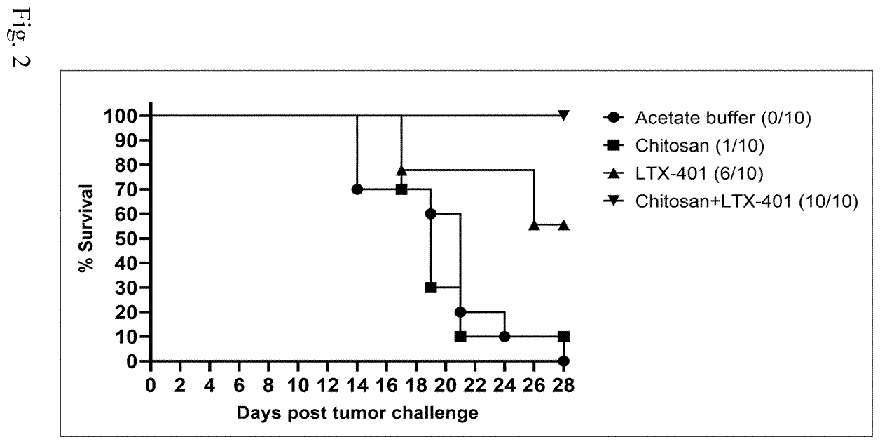

The results are shown in FIG. 2. A survival plot shows that the animals treated with composition 3 (combined composition of LTX-401 and chitosan) induced complete regression of tumors in all the treated animals, giving a survival of 100% compared to 60% in animals treated with LTX-401 alone, and 10% survival in animals treated with chitosan alone. None of the animals in the control group survived.

### EMBODIMENTS

1. Combination of a composition (a) comprising an oncolytic peptide and a composition (b) comprising chitosan and/or a chitosan derivative for use in the treatment of a subject having a tumor.

2. Composition (a) comprising an oncolytic peptide and a composition (b) comprising chitosan and/or a chitosan derivative for use in the treatment of a subject having a tumor.

3. Composition (a) which comprises an oncolytic peptide for use in the treatment of a subject having a tumor by concurrent, sequential or combined administration with a composition (b) which comprises chitosan and/or a chitosan derivative.

4. Method of treating a subject having a tumor comprising the administration to said subject of a composition (a) comprising an oncolytic peptide and a composition (b) comprising chitosan and/or a chitosan derivative.

5. Use of a composition (a) comprising an oncolytic peptide and a composition (b) comprising chitosan and/or a chitosan derivative for treating a subject having a tumor.

6. Combination, composition for use, method or use according to any of embodiments 1 to 5, wherein said compositions (a) and (b) are administered to said subject and wherein said administration is a concurrent, sequential or combined administration.

7. Combination, composition for use, method or use according to any of embodiments 1 to 6, wherein said composition (a) comprises one or more oncolytic peptides and/or wherein said composition (b) comprises one or more different chitosans and/or chitosan derivatives.

8. Combination, composition for use, method or use according to any of embodiments 1 to 7, wherein said composition (a) and/or said composition (b) comprises a physiologically acceptable carrier.

9. Combination, composition for use, method or use according to embodiment 8, wherein the physiologically acceptable carrier is selected from the group consisting of water, such as water for injection (WFI), saline and aqueous buffers.

10. Combination, composition for use, method or use according to any of embodiments 8 to 9, wherein said composition (a) and/or said composition (b) comprises a mixture of different physiologically acceptable carriers.

11. Combination, composition for use, method or use according to any of embodiments 8 to 10, wherein said composition (a) and/or said composition (b) comprises water, such as water for injection, which comprises a salt selected from the group consisting of sodium chloride, potassium chloride, calcium chloride and magnesium chloride.

12. Combination, composition for use, method or use according to any of embodiments 1 to 11, wherein the oncolytic peptide is a 9-mer peptide or a pharmaceutically acceptable salt thereof, which consists of 9 amino acids in a linear arrangement, wherein of those 9 amino acids, 5 are cationic and 4 are lipophilic, and wherein of these 4 lipophilic amino acids, 3 are tryptophan and 1 is a non-genetically coded amino acid.

13. Combination, composition for use, method or use according to embodiment 12, wherein the lipophilic and cationic amino acids are arranged such that there are no more than two of either type of amino acid adjacent to one another and/or the peptide comprises two pairs of adjacent cationic amino acids and one or two pairs of adjacent lipophilic amino acids.

14. Combination, composition for use, method or use according to any of embodiments 12 to 13, wherein the cationic amino acids are the same or different and are selected from the group consisting of lysine, arginine, histidine and a non-genetically coded amino acid carrying a positive charge at pH 7.0, preferably selected from the group consisting of lysine and arginine.

15. Combination, composition for use, method or use according to any of embodiments 12 to 14, wherein the non-genetically coded cationic amino acids are selected from the group consisting of derivatives of lysine, arginine and histidine.

16. Combination, composition for use, method or use according to any of embodiments 12 to 15, wherein the non-genetically coded cationic amino acids are selected from the group consisting of homolysine, ornithine, diaminobutyric acid, diaminopimelic acid, diaminopropionic acid, homoarginine, trimethylysine, trimethylornithine, 4-aminopiperidine-4-carboxylic acid, 4-amino-1-carbamimidoylpiperidine-4-carboxylic acid and 4-guanidinophenylalanine.

17. Combination, composition for use, method or use according to any of embodiments 12 to 16, wherein the non-genetically coded lipophilic amino acid has a lipophilic side chain with at least 7, at least 8, at least 9 or at least 10 non hydrogen atoms, preferably wherein said non hydrogen atoms are carbon atoms.

18. Combination, composition for use, method or use according to any of embodiments 12 to 17, wherein the non-genetically coded lipophilic amino acid has a lipophilic side chain that contains no more than 30 or no more than 25 non hydrogen atoms, and/or wherein such lipophilic side chain comprises at least one, preferably two optionally fused or connected cyclic groups, and/or wherein said lipophilic side chain contains heteroatoms such as O, N or S, preferably only one heteroatom, more preferably nitrogen and/or wherein said lipophilic side chain comprises 2 or less than 2 polar groups, preferably one and more preferably none.

19. Combination, composition for use, method or use according to any of embodiments 12 to 18, wherein the non-genetically coded lipophilic amino acid is selected from the group consisting of 2-amino-3-(biphenyl-4-yl) propanoic acid (biphenylalanine), 2-(diphenylalanine), 2-amino-3-(anthracen-9-amino-3,3-diphenyl-propanoic acid yl) propanoic acid, 2-amino-3-(naphthalen-2-yl) propanoic acid, 2-amino-3-(naphthalen-1-yl) propanoic acid, 2-amino-3-[1,1′: 4′,1″-terphenyl-4-yl]-propionic acid, 2-amino-3-(2,5,7-tri-tert-butyl-1H-indol-3-yl) propanoic acid, 2-amino-3-[1,1′: 3′,1″-terphenyl-4-yl]-propionic acid, 2-amino-3-[1,1′: 2′,1″-terphenyl-4-yl]-propionic acid, 2-amino-3-(4-naphthalen-2-yl-phenyl)-propionic acid, 2-amino-3-(4′-butylbiphenyl-4-yl) propanoic acid, 2-amino-3-[1,1′: 3′,1″-terphenyl-5′-yl]-propionic acid and 2-amino-3-(4-(2,2-diphenylethyl)-phenyl) propanoic acid, more preferably from the group consisting of diphenylalanine and biphenylalanine.

20. Combination, composition for use, method or use according to any of embodiments 12 to 19, wherein the 9-mer peptide is a compound of formulae (I) to (V), in which C represents a cationic amino acid and L represents a lipophilic amino acid and wherein the amino acids are covalently linked by peptide bonds and preferably wherein the carboxy terminus is amidated:

CCLLCCLLC (I),
LCCLLCCLC (II),
CLLCCLLCC (III),
CCLLCLLCC (IV),
CLCCLLCCL (V).

21. Combination, composition for use, method or use according to any of embodiments 12 to 20, wherein the 9-mer peptide is a compound of formulae (I′), (I″), (1″) or (II′), in which C represents a cationic amino acid and L′ represents a non-genetically coded lipophilic amino acid and wherein the amino acids are covalently linked by peptide bonds and preferably wherein the carboxy terminus is amidated:

CCL'LCCLLC (I'),
CCLLCCLL'C (I″),
CCLL'CCLLC (I″),
LCCLL'CCLC (II').

22. Combination, composition for use, method or use according to any of embodiments 1 to 21, wherein the oncolytic peptide is selected from the group consisting of LTX-301, LTX-302, LTX-303, LTX-304, LTX-305, LTX-306, LTX-307, LTX-308, LTX-309, LTX-310, LTX-310, LTX-311, LTX-312, LTX-313, LTX-314, LTX-315, LTX-316, LTX-317, LTX-318, LTX-319, LTX-320, LTX-321, LTX-322, LTX-323, LTX-324, LTX-325, LTX-326, LTX-327, LTX-328, LTX-329, LTX-330, LTX-331, LTX-332, LTX-333, LTX-334, LTX-335, LTX-336, LTX-337 and LTX-338.

23. Combination, composition for use, method or use according to any of embodiments 1 to 22, wherein the oncolytic peptide is selected from the group consisting of LTX-302, LTX-313, LTX-315, LTX-320 and LTX-329, preferably wherein the oncolytic peptide is LTX-315.

24. Combination, composition for use, method or use according to any of embodiments 1 to 23, wherein the oncolytic peptide is LTX-315 in the form of a pharmaceutically acceptable salt, preferably in the form of an acetate salt.

25. Combination, composition for use, method or use according to any of embodiments 1 to 24, wherein composition (a) further comprises one or more preservative agents and/or one or more chelating agents and/or one or more antioxidants and/or one or more tonicity modifiers and/or one or more stabilizers and/or one or more viscosity enhancers.

26. Combination, composition for use, method or use according to any of embodiments 1 to 11, wherein the oncolytic peptide is a peptide, peptidomimetic or amino acid derivative having a net positive charge of at least +2 and incorporating a disubstituted β amino acid, wherein each of the substituting groups in the β amino acid comprises at least 7 non-hydrogen atoms, is lipophilic and has at least one cyclic group, wherein one or more cyclic groups within a substituting group are optionally fused to one or more cyclic groups within the other substituting group.

27. Combination, composition for use, method or use according to embodiment 26, wherein one or more cyclic groups within a substituting group are fused to one or more cyclic groups within the other substituting group and the combined total number of non-hydrogen atoms for the two substituting groups is at least 12.

28. Combination, composition for use, method or use according to any of embodiments 26 to 27, wherein the oncolytic peptide is a compound that incorporates a group of formula (VI)

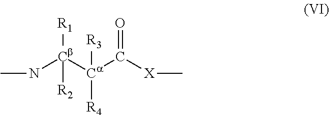

wherein X and N have their normal valencies and are bound to other parts of said compound and wherein any 2 from R₁, R₂, R₃ and R₄ are hydrogen atoms and 2 are substituting groups, which comprise at least 7 non-hydrogen atoms, are lipophilic and include a cyclic group, wherein said cyclic group is not attached directly either to the α or β carbon atom and is optionally linked or fused to a cyclic group in the other substituting group, and wherein X represents O, C, N or S.

29. Combination, composition for use, method or use according to embodiment 28, wherein said substituting groups are the same or are different.

30. Combination, composition for use, method or use according to any of embodiments 28 or 29, wherein said cyclic group is linked or fused to a cyclic group in the other substituting group and wherein the combined total number of non-hydrogen atoms for the two substituting groups is at least 12.

31. Combination, composition for use, method or use according to any of embodiments 28 or 30, wherein the nitrogen atom in the group of formula (VI) is not bound to any atom of groups R₁₋₄, and/or wherein the 5 atoms in the backbone (N-C^{β}-C^{α}-C-X) are connected to each other in a linear fashion and/or wherein R₁₋₄ are lipophilic in nature and preferably carry no charge and preferably have no more than two, more preferably no more than one polar group and/or wherein X is a substituted N-atom.

32. Combination, composition for use, method or use according to any of embodiments 28 or 31, wherein the compound incorporating a group of formula VI have an amidated C-terminus.

33. Combination, composition for use, method or use according to any of embodiments 1 to 11 and 26 to 32, wherein the oncolytic peptide is LTX-401, i.e. the compound of formula (VII)

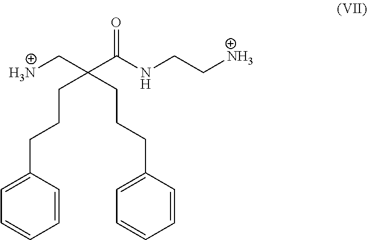 [SMILES: [NH3+]CCNC(=O)C(C[NH3+])(CCCc1ccccc1)CCCc1ccccc1]

34. Combination, composition for use, method or use according to embodiment 33, wherein LTX 401 is in the form of a pharmaceutically acceptable salt, preferably in the form of a hydrochloride or acetate salt.

35. Combination, composition for use, method or use according to any of the preceding embodiments, wherein composition (a) comprises the oncolytic peptide at a concentration of from 0.1 to 30 mg/ml, such as 1 to 5 mg/ml or 1 to 30 mg/ml, preferably 4 to 25 mg/ml, or more preferably 6 to 20 mg/ml.

36. Combination, composition for use, method or use according to any of the preceding embodiments, wherein composition (a) has a pH of from 4.0 to 7.4, more preferably of from 5.0 to 7.0, and even more preferably of from 5.0 to 6.5, such as 6.0.

37. Combination, composition for use, method or use according to any of the preceding embodiments, wherein the oncolytic peptide is in the form of a pharmaceutically acceptable salt, more preferably in the form of a hydrochloride or acetate salt.

38. Combination, composition for use, method or use according to any of the preceding embodiments, wherein composition (b) comprises chitosan.

39. Combination, composition for use, method or use according to any of the preceding embodiments, wherein composition (b) comprises several different chitosans.

40. Combination, composition for use, method or use according to any of embodiments 38 to 39, wherein said chitosan is a very low molecular weight chitosan, low molecular weight chitosan and/or a medium molecular weight chitosan.

41. Combination, composition for use, method or use according to any of the preceding embodiments, wherein composition (b) comprises a chitosan derivative.

42. Combination, composition for use, method or use according to embodiment 41, wherein composition (b) comprises several different chitosan derivatives.

43. Combination, composition for use, method or use according to any of embodiments 41 to 42, wherein said chitosan derivative is a glycated chitosan, preferably a glycated chitosan which possesses from about 0.1% to about 90% percent glycation of its otherwise free amino groups and/or which has a molecular weight from about 50 kD to about 2000 kD.

44. Combination, composition for use, method or use according to any of the preceding embodiments, wherein the chitosan and/or chitosan derivative has a degree of deacetylation (DDA) of about 50 to 99%, preferably of about 70% to about 99%.

45. Combination, composition for use, method or use according to any of the preceding embodiments, wherein composition (b) comprises 0.1 to 5% w/w of the chitosan and/or the chitosan derivative, such as 0.1 to 2.5% w/w, such as 0.5%, 1%, 1.5% or 2% w/w.

46. Combination, composition for use, method or use according to any of the preceding embodiments, wherein composition (b) is a dispersion, suspension or solution of the chitosan and/or the chitosan derivative in a physiologically acceptable carrier.

47. Combination, composition for use, method or use according to any of the preceding embodiments, wherein composition (b) has a pH of from 4.0 to 7.5, preferably of from 5.0 to 7.4 and more preferably of from 5.0 to 7.0, such as 6.5 or 6.0.

48. Combination, composition for use, method or use according to any of the preceding embodiments, wherein composition (a) and/or (b) has a viscosity of from 1.0, 1.5, 2, 2.5 or 3 cP to 300 cP, e.g. from 2.5 to 250 cP, such as 5 to 100 cP or 10 to 200 cP and 20 to 240 cP, preferably of from 40 to 180 cP, e.g. from 45 to 150 cP or from 30 to 100 cP and more preferably 1.5 to 10 cP, such as 2, 3, 4, 5, 6, 7, 8, 9 or 10 cP or of from 10 to 15 cP or 15 to 20 cP or 20 to 35 cP or 35 to 50 cP or 50 to 100 cP.

49. Combination, composition for use, method or use according to any of the preceding embodiments, wherein composition (a) is administered into said tumor, such as administered into the tumor by injection and/or by perfusion/infusion.

50. Combination, composition for use, method or use according to any of the preceding embodiments, wherein composition (b) is administered into said tumor, such as administered into the tumor by injection and/or by perfusion/infusion and/or administered into the tissue immediately surrounding the tumor.

51. Combination, composition for use, method or use according to any of the preceding embodiments, wherein composition (a) and composition (b) are administered concurrently, such as simultaneously (e.g. by the same or different way of administration) or within a short period of time, such as within minutes or hours or longer, but on the same day.

52. Combination, composition for use, method or use according to embodiment 51, wherein composition (a) is administered first, followed by administration of composition (b).

53. Combination, composition for use, method or use according to embodiment 51, wherein composition (b) is administered first, followed by administration of composition (a).

54. Combination, composition for use, method or use according to any of embodiments 1 to 50, wherein composition (a) and composition (b) are administered sequentially, such as on different days (e.g. by the same or different way of administration), such as with a day or days apart or such as with a week or weeks apart.

55. Combination, composition for use, method or use according to embodiment 54, wherein composition (a) is administered first, followed by administration of composition (b).

56. Combination, composition for use, method or use according to embodiment 54, wherein composition (b) is administered first, followed by administration of composition (a).

57. Combination, composition for use, method or use according to any of the preceding embodiments, wherein each of composition (a) and b) are administered, such as administered into the tumor, only once.

58. Combination, composition for use, method or use according to any of embodiments 1 to 56, wherein each of composition (a) and (b) are administered, such as administered into the tumor, repeatedly.

59. Combination, composition for use, method or use according to any of embodiments 1 to 56, wherein one of the composition (a) and (b) are administered, such as administered into the tumor, only once and the other composition is administered, such as administered into the tumor, repeatedly.

60. Combination, composition for use, method or use according to any of the preceding embodiments, wherein composition (a) and composition (b) are administered, e.g. administered into the tumor, in a therapeutically effective amount and wherein such amount is administered in one administration or in several administrations.

61. Combination, composition for use, method or use according to any of the preceding embodiments, wherein said tumor is a tumor selected from the group consisting of tumors of breast cancer, ovarian cancer, colon cancer, prostate cancer, bone cancer, colorectal cancer, gastric cancer, skin cancer, small cell lung cancer, non-small cell lung cancer, pancreatic cancer, thyroid cancers, kidney cancer, liver cancer, cancer of the bile duct, brain cancer, cervical cancer, bladder cancer, esophageal cancer; Hodgkin's disease and adrenocortical cancer.

62. Combination, composition for use, method or use according to any of the preceding embodiments, wherein said tumor is a tumor selected from the group consisting of carcinoma, adenocarcinoma, lymphoma, melanoma, blastoma, leukemia, sarcoma and germ cell tumor.

63. Combination, composition for use, method or use according to any of the preceding embodiments, wherein composition (a) and composition (b) are pharmaceutical compositions.

64. Composition comprising i) an oncolytic peptide and ii) chitosan and/or a chitosan derivative.

65. Composition according to embodiment 64, comprising one or more oncolytic peptides and/or comprising one or more different chitosans and/or chitosan derivatives.

66. Composition according to any one of embodiments 64 to 65, wherein said composition comprises a physiologically acceptable carrier.

67. Composition according to embodiment 66, wherein the physiologically acceptable carrier is selected from the group consisting of water, such as water for injection (WFI), saline and aqueous buffers.

68. Composition according to any of embodiments 66 to 67, wherein said composition comprises a mixture of different physiologically acceptable carriers.

69. Composition according to any of embodiments 66 to 68, wherein said composition comprises water, such as water for injection, comprises a salt selected from the group consisting of sodium chloride, potassium chloride, calcium chloride and magnesium chloride.

70. Composition according to any of embodiments 64 to 69, wherein the oncolytic peptide is a 9-mer peptide or a pharmaceutically acceptable salt thereof, which consists of 9 amino acids in a linear arrangement, wherein of those 9 amino acids, 5 are cationic and 4 are lipophilic, and wherein of these 4 lipophilic amino acids, 3 are tryptophan and 1 is a non-genetically coded amino acid.

71. Composition according to embodiment 70, wherein the lipophilic and cationic amino acids are arranged such that there are no more than two of either type of amino acid adjacent to one another and/or the peptide comprises two pairs of adjacent cationic amino acids and one or two pairs of adjacent lipophilic amino acids.

72. Composition according to any of embodiments 70 to 71, wherein the cationic amino acids are the same or different and are selected from the group consisting of lysine, arginine, histidine and a non-genetically coded amino acid carrying a positive charge at pH 7.0, preferably selected from the group consisting of lysine and arginine.

73. Composition according to any of embodiments 70 to 72, wherein the non-genetically coded cationic amino acids are selected from the group consisting of derivatives of lysine, arginine and histidine.

74. Composition according to any of embodiments 70 to 73, wherein the non-genetically coded cationic amino acids are selected from the group consisting of homolysine, ornithine, diaminobutyric acid, diaminopimelic acid, diaminopropionic acid, homoarginine, trimethylysine, trimethylornithine, 4-aminopiperidine-4-carboxylic acid, 4-amino-1-carbamimidoylpiperidine-4-carboxylic acid and 4-guanidinophenylalanine.

75. Composition according to any of embodiments 70 to 74, wherein the non-genetically coded lipophilic amino acid has a lipophilic side chain with at least 7, at least 8, at least 9 or at least 10 non hydrogen atoms, preferably wherein said non hydrogen atoms are carbon atoms.

76. Composition according to any of embodiments 70 to 75, wherein the non-genetically coded lipophilic amino acid has a lipophilic side chain that contains no more than 30 or no more than 25 non hydrogen atoms, and/or wherein such lipophilic side chain comprises at least one, preferably two optionally fused or connected cyclic groups, and/or wherein said lipophilic side chain contains heteroatoms such as O, N or S, preferably only one heteroatom, more preferably nitrogen and/or wherein said lipophilic side chain comprises 2 or less than 2 polar groups, preferably one and more preferably none.

77. Composition according to any of embodiments 70 to 76, wherein the non-genetically coded lipophilic amino acid is selected from the group consisting of 2-amino-3-(biphenyl-4-yl) propanoic acid (biphenylalanine), 2-amino-3,3-diphenyl-propanoic acid (diphenylalanine), 2-amino-3-(anthracen-9-yl) propanoic acid, 2-amino-3-(naphthalen-2-yl) propanoic acid, 2-amino-3-(naphthalen-1-yl) propanoic acid, 2-amino-3-[1,1′: 4′,1″-terphenyl-4-yl]-propionic acid, 2-amino-3-(2,5,7-tri-tert-butyl-1H-indol-3-yl) propanoic acid, 2-amino-3-[1,1′: 3′,1″-terphenyl-4-yl]-propionic acid, 2-amino-3-[1,1′: 2′,1″-terphenyl-4-yl]-propionic acid, 2-amino-3-(4-naphthalen-2-yl-phenyl)-propionic acid, 2-amino-3-(4′-butylbiphenyl-4-yl) propanoic acid, 2-amino-3-[1,1′: 3′,1″-terphenyl-5′-yl]-propionic acid and 2-amino-3-(4-(2,2-diphenylethyl)-phenyl) propanoic acid, more preferably from the group consisting of diphenylalanine and biphenylalanine.

78. Composition according to any of embodiments 70 to 77, wherein the 9-mer peptide is a compound of formulae (I) to (V), in which C represents a cationic amino acid and L represents a lipophilic amino acid and wherein the amino acids are covalently linked by peptide bonds and preferably wherein the carboxy terminus is amidated:

CCLLCCLLC (I),
LCCLLCCLC (II),
CLLCCLLCC (III),
CCLLCLLCC (IV),
CLCCLLCCL (V).

79. Composition according to any of embodiments 70 to 78, wherein the 9-mer peptide is a compound of formulae (I′), (I″), (I″) or (II′), in which C represents a cationic amino acid and L′ represents a non-genetically coded lipophilic amino acid and wherein the amino acids are covalently linked by peptide bonds and preferably wherein the carboxy terminus is amidated:

CCL'LCCLLC (I'),
CCLLCCLL'C (I″),
CCLL'CCLLC (I″),
LCCLL'CCLC (II').

80. Composition according to any of embodiments 70 to 79, wherein the oncolytic peptide is selected from the group consisting of LTX-301, LTX-302, LTX-303, LTX-304, LTX-305, LTX-306, LTX-307, LTX-308, LTX-309, LTX-310, LTX-310, LTX-311, LTX-312, LTX-313, LTX-314, LTX-315, LTX-316, LTX-317, LTX-318, LTX-319, LTX-320, LTX-321, LTX-322, LTX-323, LTX-324, LTX-325, LTX-326, LTX-327, LTX-328, LTX-329, LTX-330, LTX-331, LTX-332, LTX-333, LTX-334, LTX-335, LTX-336, LTX-337 and LTX-338.

81. Composition according to any of embodiments 70 to 80, wherein the oncolytic peptide is selected from the group consisting of LTX-302, LTX-313, LTX-315, LTX-320 and LTX-329, preferably wherein the oncolytic peptide is LTX-315.

82. Composition according to any of embodiments 70 to 81, wherein the oncolytic peptide is LTX-315 in the form of a pharmaceutically acceptable salt, preferably in the form of an acetate salt.

83. Composition according to any of embodiments 70 to 82, wherein composition (a) further comprises one or more preservative agents and/or one or more chelating agents and/or one or more antioxidants and/or one or more tonicity modifiers and/or one or more stabilizers and/or one or more viscosity enhancers.

84. Composition according to any of embodiments 61 to 63, wherein the oncolytic peptide is a peptide, peptidomimetic or amino acid derivative having a net positive charge of at least +2 and incorporating a disubstituted β amino acid, wherein each of the substituting groups in the β amino acid comprises at least 7 non-hydrogen atoms, is lipophilic and has at least one cyclic group, wherein one or more cyclic groups within a substituting group are optionally fused to one or more cyclic groups within the other substituting group.

85. Composition according to embodiment 84, wherein one or more cyclic groups within a substituting group are fused to one or more cyclic groups within the other substituting group and the combined total number of non-hydrogen atoms for the two substituting groups is at least 12.

86. Composition according to any of embodiments 84 to 85, wherein the oncolytic peptide is a compound that incorporates a group of formula (IV)

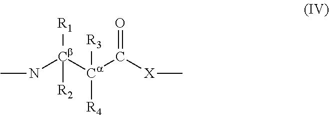

wherein X and N have their normal valencies and are bound to other parts of said compound and wherein any 2 from R₁, R₂, R₃ and R₄ are hydrogen atoms and 2 are substituting groups, which comprise at least 7 non-hydrogen atoms, are lipophilic and include a cyclic group, wherein said cyclic group is not attached directly either to the α or β carbon atom and is optionally linked or fused to a cyclic group in the other substituting group, and wherein X represents O, C, N or S.

87. Composition according to embodiment 86, wherein said substituting groups are the same or are different.

88. Composition according to any of embodiments 86 to 87, wherein said cyclic group is linked or fused to a cyclic group in the other substituting group and wherein the combined total number of non-hydrogen atoms for the two substituting groups is at least 12.

89. Composition according to any of embodiments 86 to 88, wherein the nitrogen atom in the group of formula (VI) is not bound to any atom of groups R₁₋₄, and/or wherein the 5 atoms in the backbone (N-C^{β}-C^{α}-C-X) are connected to each other in a linear fashion and/or wherein R¹⁴ are lipophilic in nature and preferably carry no charge and preferably have no more than two, more preferably no more than one polar group and/or wherein X is a substituted N-atom.

90. Composition according to any of embodiments 86 to 89, wherein the compounds incorporating a group of formula VI have an amidated C-terminus.

91. Composition according to any of embodiments 64 to 90, wherein the oncolytic peptide is LTX-401, i.e. the compound of formula (VII)

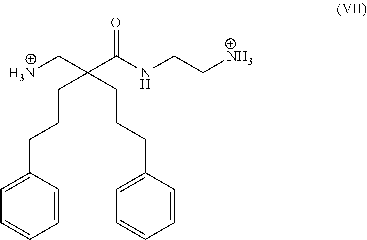 [SMILES: [NH3+]CCNC(=O)C(C[NH3+])(CCCc1ccccc1)CCCc1ccccc1]

92. Composition according to embodiment 91, wherein LTX 401 is in the form of a pharmaceutically acceptable salt, preferably in the form of a hydrochloride or acetate salt.

93. Composition according to any one of embodiments 64 to 92, wherein the oncolytic peptide is present at a concentration of from 0.1 to 30 mg/ml, such as 1 to 5 mg/ml or 1 to 30 mg/ml, preferably 4 to 25 mg/ml, or more preferably 6 to 20 mg/ml.

94. Composition according to any one of embodiments 64 to 93, wherein the oncolytic peptide is in the form of a pharmaceutically acceptable salt, more preferably in the form of a hydrochloride or acetate salt.

95. Composition according to any one of embodiments 64 to 94, wherein said composition comprises chitosan.

96. Composition according to any one of embodiments 64 to 95, wherein said composition comprises several different chitosans.

97. Composition according to any one of embodiments 64 to 96, wherein said chitosan is a very low molecular weight chitosan, low molecular weight chitosan and/or a medium molecular weight chitosan.

98. Composition according to any one of embodiments 64 to 97, wherein said composition comprises a chitosan derivative.

99. Composition according to embodiment 98, wherein said composition comprises several different chitosan derivatives.

100. Composition according to any one of embodiments 64 to 99, wherein said chitosan derivative is a glycated chitosan, preferably a glycated chitosan which possesses from about 0.1% to about 90% percent glycation of its otherwise free amino groups and/or which has a molecular weight from about 50 kD to about 2000 kD.

101. Composition according to any one of embodiments 64 to 100, wherein the chitosan and/or chitosan derivative has a degree of deacetylation (DDA) of about 50 to 99%, preferably of about 70% to about 99%.

102. Composition according to any one of embodiments 64 to 101, wherein said composition comprises 0.1 to 5% w/w of the chitosan and/or the chitosan derivative, such as 0.1 to 2.5% w/w, such as 0.5%, 1%, 1.5% or 2% w/w.

103. Composition according to any one of embodiments 64 to 102, wherein the composition is a dispersion, suspension or a solution of i) and ii) in a physiologically acceptable carrier.

104. Composition according to any one of embodiments 64 to 103, wherein the composition has a pH of from 4.0 to 7.4, more preferably of from 5.0 to 7.0, such as 6.0 or 6.5.

105. Composition according to any one of embodiments 64 to 104, wherein the composition has a viscosity of from 1.0, 1.5, 2, 2.5 or 3 cP to 300 cP, e.g. from 2.5 to 250 cP, such as 5 to 100 cP or 10 to 200 cP and 20 to 240 cP, preferably of from 40 to 180 cP, e.g. from 45 to 150 cP or from 30 to 100 cP and more preferably 1.5 to 10 cP, such as 2, 3, 4, 5, 6, 7, 8, 9 or 10 cP or of from 10 to 15 cP or 15 to 20 cP or 20 to 35 cP or 35 to 50 cP or 50 to 100 cP.

106. Composition according to any one of embodiments 64 to 105, wherein the composition is a pharmaceutical composition.

107. Composition according to any one of embodiments 64 to 106 contained in a sealed container.

108. Composition as defined in any one of embodiments 64 to 107 for use as a medicament.

109. Composition as defined in any one of embodiments 64 to 108 for use in a method of treating a tumor in a subject.

110. Composition for use according to embodiment 109, wherein the composition is administered to the subject, such as administered into the tumor.

111. Composition for use according to embodiment 110, wherein said composition is administered, such as administered into the tumor, only once.

112. Composition for use according to embodiment 110, wherein said composition is administered, such as administered into the tumor, repeatedly.

113. Composition for use according to any of embodiments 109 to 112, wherein said composition is administered, e.g. administered into the tumor, in a therapeutically effective amount and wherein such amount is administered in one administration or in several administrations.

114. Composition for use according to any of embodiments 109 to 113, wherein said tumor is selected from the group consisting of tumors of breast cancer, ovarian cancer, colon cancer, prostate cancer, bone cancer, colorectal cancer, gastric cancer, skin cancer, small cell lung cancer, non-small cell lung cancer, pancreatic cancer, thyroid cancers, kidney cancer, liver cancer, cancer of the bile duct, brain cancer, cervical cancer, bladder cancer, esophageal cancer; Hodgkin's disease and adrenocortical cancer.

115. Composition for use according to any of embodiments 109 to 114, wherein said tumor is a tumor selected from the group consisting of carcinoma, adenocarcinoma, lymphoma, melanoma, blastoma, leukemia, sarcoma and germ cell tumor.

116. Method for preparing a composition as defined in any of embodiments 64 to 106, wherein chitosan and/or a chitosan derivative is dispersed, suspended or dissolved in a physiologically acceptable carrier and the oncolytic peptide is dissolved in said dispersion, suspension or solution or wherein chitosan and/or a chitosan derivative is dispersed, suspended or dissolved in a solution of the oncolytic peptide in the physiologically acceptable carrier.

117. Method according to embodiment 116, wherein the composition is sterile filtered and then filled into a container and sealed.

118. Method according to embodiment 116, wherein the composition is filled into a container, sealed and autoclaved.

119. Kit comprising a composition (a) and (b) as defined in any of embodiments 1 to 63 or comprising parts/components to prepare said compositions (a) and (b).

120. Kit according to embodiment 119, comprising the oncolytic peptide and a physiologically acceptable carrier to prepare composition (a) and the chitosan and/or a chitosan derivative and a physiologically acceptable carrier to prepare composition (b) and optionally comprising instructions for preparing compositions (a) and (b).

121. Kit according to embodiment 120, wherein said physiologically acceptable carrier for preparing composition (a) is the same as the physiologically acceptable carrier for preparing composition (b).

122. Kit according to embodiment 119, comprising composition (a), the chitosan and/or a chitosan derivative (e.g. in solid form) and a physiologically acceptable carrier to prepare composition (b) and optionally comprising instructions for preparing composition (b).

123. Kit according to embodiment 119, comprising composition (b), the oncolytic peptide (e.g. in solid form) and a physiologically acceptable carrier to prepare composition (a) and optionally comprising instructions for preparing composition (a).

124. Kit according to any of embodiments 119 to 123, further comprising instructions for use of compositions (a) and (b).

125. Kit according to embodiment 119, comprising a composition (a) and (b) as defined in any of embodiments 1 to 63 and instructions for preparing a composition as defined in any of embodiments 64 to 106 by combining said composition (a) and said composition (b).

126. Kit according to embodiment 125, further comprising instructions for use of said combined composition.

127. Kit comprising parts/components to prepare the composition as defined in any of embodiments 64 to 106.

128. Kit according to embodiment 127, wherein the kit comprises the oncolytic peptide, the chitosan and/or a chitosan derivative (e.g. both in solid form) and a physiologically acceptable carrier to prepare the combined composition.

129. Kit according to embodiment 128, wherein the oncolytic peptide, the chitosan and/or a chitosan derivative are comprised in one container.

130. Kit according to embodiment 128, wherein the oncolytic peptide and the chitosan and/or a chitosan derivative are comprised in separate containers.

131. Kit according to embodiment 127, wherein the kit comprises composition (a) comprising as defined in any of embodiments 1 to 63 as defined in any of embodiments 1 to 63 and the chitosan and/or a chitosan derivative (e.g. in solid form) and optionally comprising instructions for preparing the composition as defined in any of embodiments 64 to 106.

132. Kit according to embodiment 127, wherein the kit comprises composition (b) as defined in any of embodiments 1 to 63 comprising a physiologically acceptable carrier and the oncolytic peptide (e.g. in solid form) and optionally comprising instructions for preparing the composition as defined in any of embodiments 64 to 106.

133. Kit according to any of embodiments 127 to 132, further comprising instructions for use of the composition as defined in any of embodiments 64 to 106.

1. Composition (a) which comprises an oncolytic peptide for use in the treatment of a subject having a tumor by concurrent, sequential or combined administration with a composition (b) which comprises chitosan and/or a chitosan derivative.

2. Composition for use as claimed in claim 1, wherein said composition (a) comprises one or more oncolytic peptides and/or wherein said composition (b) comprises one or more different chitosans and/or chitosan derivatives.

3. Composition for use as claimed in any of claims 1 to 2, wherein said composition (a) and/or said composition (b) comprises a physiologically acceptable carrier.

4. Composition for use as claimed in any of claims 1 to 3, wherein the oncolytic peptide is a 9-mer peptide or a pharmaceutically acceptable salt thereof, which consists of 9 amino acids in a linear arrangement, wherein of those 9 amino acids, 5 are cationic and 4 are lipophilic, and wherein of these 4 lipophilic amino acids, 3 are tryptophan and 1 is a non-genetically coded amino acid.

5. Composition for use as claimed in claim 4, wherein the cationic amino acids are lysine or arginine and/or wherein the non-genetically coded amino acid is diphenylalanine or biphenylalanine.

6. Composition for use as claimed in any of claims 1 to 5, wherein the oncolytic peptide is selected from the group consisting of LTX-302, LTX-313, LTX-315, LTX-320 and LTX-329.

7. Composition for use as claimed in any of claims 1 to 6, wherein the oncolytic peptide is LTX-315.

8. Composition for use as claimed in any of claims 1 to 7, wherein composition (a) further comprises one or more preservative agents and/or one or more chelating agents and/or one or more antioxidants and/or one or more tonicity modifiers and/or one or more stabilizers and/or one or more viscosity enhancers.

9. Composition for use as claimed in any of claims 1 to 3, wherein the oncolytic peptide is a peptide, peptidomimetic or amino acid derivative having a net positive charge of at least +2 and incorporating a disubstituted β amino acid, wherein each of the substituting groups in the β amino acid comprises at least 7 non-hydrogen atoms, is lipophilic and has at least one cyclic group, wherein one or more cyclic groups within a substituting group are optionally fused to one or more cyclic groups within the other substituting group.

10. Composition for use as claimed in claim 9, wherein the oncolytic peptide is a compound that incorporates a group of formula (VI) 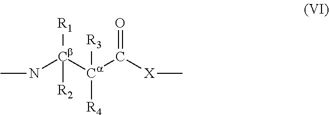
wherein X and N have their normal valencies and are bound to other parts of said compound and wherein any 2 from R₁, R₂, R₃ and R₄ are hydrogen atoms and 2 are substituting groups, which comprise at least 7 non-hydrogen atoms, are lipophilic and include a cyclic group, wherein said cyclic group is not attached directly either to the α or β carbon atom and is optionally linked or fused to a cyclic group in the other substituting group, and wherein X represents O, C, N or S.

11. Composition for use as claimed in claim 10, wherein the incorporating a group of formula (VI) have an amidated C-terminus.

12. Composition for use as claimed in any of claims 9 to 11, wherein the oncolytic peptide is LTX-401, i.e. the compound of formula (VII)

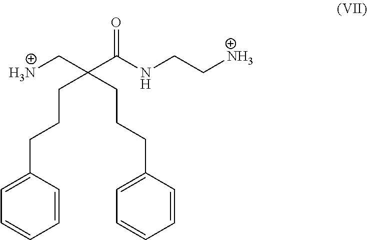 [SMILES: [NH3+]CCNC(=O)C(C[NH3+])(CCCc1ccccc1)CCCc1ccccc1]

13. Composition for use as claimed in any of the preceding claims, wherein the oncolytic peptide is in the form of a pharmaceutically acceptable salt.

14. Composition for use as claimed in any of the preceding claims, wherein composition (a) comprises the oncolytic peptide at a concentration of from 0.1 to 30 mg/ml.

13. Composition for use as claimed in any of the preceding claims, wherein composition (a) has a pH of from 4.0 to 7.4.

14. Composition for use as claimed in any of the preceding claims, wherein composition (b) comprises chitosan.

15. Composition for use as claimed in any of the preceding claims, wherein composition (b) comprises a chitosan derivative.

16. Composition for use as claimed in claim 15, wherein said chitosan derivative is glycated chitosan.

17. Composition for use as claimed in any of the preceding claims, wherein the chitosan and/or chitosan derivative has a degree of deacetylation (DDA) of about 50 to 99%.

18. Composition for use as claimed in any of the preceding claims, wherein composition (b) comprises 0.1 to 5% w/w of the chitosan and/or the chitosan derivative.

19. Composition for use as claimed in any of the preceding claims, wherein composition (b) comprises a very low molecular weight, low molecular weight or medium molecular weight chitosan and/or chitosan derivative.

20. Composition for use as claimed in any of the preceding claims, wherein composition (b) is a dispersion, suspension or solution of the chitosan and/or the chitosan derivative in a physiologically acceptable carrier.

21. Composition for use as claimed in any of the preceding claims, wherein composition (b) has a pH of from 4.0 to 7.5.

22. Composition for use as claimed in any of the preceding claims, wherein composition (a) and/or composition (b) has a viscosity of from 1.0 to 300 cP.

23. Composition for use as claimed in any of the preceding claims, wherein composition (a) is administered into the tumor and composition (b) is administered into the tumor and/or into the tissue immediately surrounding the tumor.

24. Composition for use as claimed in any of the preceding claims, wherein said tumor is a tumor selected from the group consisting of tumors of breast cancer, ovarian cancer, colon cancer, prostate cancer, bone cancer, colorectal cancer, gastric cancer, skin cancer, small cell lung cancer, non-small cell lung cancer, pancreatic cancer, thyroid cancers, kidney cancer, liver cancer, cancer of the bile duct, brain cancer, cervical cancer, bladder cancer, esophageal cancer; Hodgkin's disease and adrenocortical cancer.

25. Composition comprising i) an oncolytic peptide and ii) chitosan and/or a chitosan derivative.

26. Composition as claimed in claim 25, comprising one or more oncolytic peptides and/or comprising one or more different chitosans and/or chitosan derivatives.

27. Composition as claimed in any one of claims 25 to 26, wherein said composition comprises a physiologically acceptable carrier.

28. 4. Composition as claimed in any of claims 25 to 27, wherein the oncolytic peptide is a 9-mer peptide or a pharmaceutically acceptable salt thereof, which consists of 9 amino acids in a linear arrangement, wherein of those 9 amino acids, 5 are cationic and 4 are lipophilic, and wherein of these 4 lipophilic amino acids, 3 are tryptophan and 1 is a non-genetically coded amino acid.

29. Composition as claimed in claim 28, wherein the cationic amino acids are lysine or arginine and/or wherein the non-genetically coded amino acid is diphenylalanine or biphenylalanine.

30. Composition as claimed in any of claims 25 to 29, wherein the oncolytic peptide is selected from the group consisting of LTX-302, LTX-313, LTX-315, LTX-320 and LTX-329.

31. Composition as claimed in any of claims 25 to 30, wherein the oncolytic peptide is LTX-315.

32. Composition as claimed in any of claims 25 to 31, further comprising one or more preservative agents and/or one or more chelating agents and/or one or more antioxidants and/or one or more tonicity modifiers and/or one or more stabilizers and/or one or more viscosity enhancers.

33. Composition as claimed in any of claims 25 to 27, wherein the oncolytic peptide is a peptide, peptidomimetic or amino acid derivative having a net positive charge of at least +2 and incorporating a disubstituted β amino acid, wherein each of the substituting groups in the β amino acid comprises at least 7 non-hydrogen atoms, is lipophilic and has at least one cyclic group, wherein one or more cyclic groups within a substituting group are optionally fused to one or more cyclic groups within the other substituting group.

34. Composition as claimed in claim 33, wherein the oncolytic peptide is a compound that incorporates a group of formula (VI) 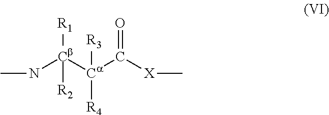
wherein X and N have their normal valencies and are bound to other parts of said compound and wherein any 2 from R₁, R₂, R₃ and R₄ are hydrogen atoms and 2 are substituting groups, which comprise at least 7 non-hydrogen atoms, are lipophilic and include a cyclic group, wherein said cyclic group is not attached directly either to the α or β carbon atom and is optionally linked or fused to a cyclic group in the other substituting group, and wherein X represents O, C, N or S.

35. Composition as claimed in claim 34, wherein the incorporating a group of formula (VI) have an amidated C-terminus.

36. Composition as claimed in any of claims 33 to 35, wherein the oncolytic peptide is LTX-401, i.e. the compound of formula (VII)

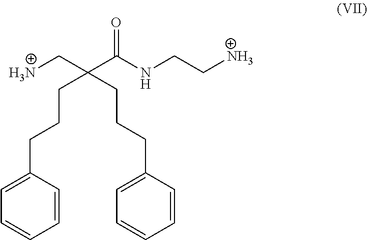 [SMILES: [NH3+]CCNC(=O)C(C[NH3+])(CCCc1ccccc1)CCCc1ccccc1]

37. Composition as claimed in any of claims 25 to 36, wherein the oncolytic peptide is in the form of a pharmaceutically acceptable salt.

38. Composition as claimed in any of claims 25 to 37, comprising the oncolytic peptide at a concentration of from 0.1 to 30 mg/ml.

39. Composition as claimed in any of claims 25 to 38, comprising chitosan.

40. Composition as claimed in any of claims 25 to 39, comprising a chitosan derivative.

41. Composition as claimed in claim 40, wherein said chitosan derivative is glycated chitosan.

42. Composition as claimed in any of claims 25 to 41, wherein the chitosan and/or chitosan derivative has a degree of deacetylation (DDA) of about 50 to 99%.

43. Composition as claimed in any of the claims 25 to 42, comprising 0.1 to 5% w/w of the chitosan and/or the chitosan derivative.

44. Composition as claimed in any of claims 25 to 43, comprising a very low molecular weight, low molecular weight or medium molecular weight chitosan and/or chitosan derivative.

45. Composition as claimed in any of claims 25 to 44, wherein the composition is a dispersion, suspension or a solution of i) and ii) in a physiologically acceptable carrier.

46. Composition as claimed in any of claims 25 to 45, wherein the composition has a pH of from 4.0 to 7.4.

47. Composition as claimed in any of claims 25 to 46, wherein the composition has a viscosity of from 1.0 to 300 cP.

48. Composition as defined in any of claims 25 to 47 for use as a medicament.

49. Composition as defined in any of claims 25 to 47 for use in a method of treating a tumor in a subject.

50. Method for preparing a composition as defined in any of claims 25 to 47, wherein chitosan and/or a chitosan derivative is dispersed, suspended or dissolved in a physiologically acceptable carrier and the oncolytic peptide is dissolved in said dispersion, suspension or solution or wherein chitosan and/or a chitosan derivative is dispersed, suspended or dissolved in a solution of the oncolytic peptide in the physiologically acceptable carrier.

51. Kit comprising a composition (a) and a composition (b) as defined in any of claims 1 to 24 or comprising parts/components to prepare said compositions (a) and (b).

52. Kit as claimed in claim 51, comprising a composition (a) and a composition (b) as defined in any of claims 1 to 24 and instructions for preparing a composition as defined in any of claims 25 to 47 by combining said composition (a) and said composition (b).

53. Kit comprising parts/components to prepare the composition as defined in any of claims 25 to 47.


SEQUENCE LISTING (42 sequences)

SEQ ID NO: 1 - protein, 9 aa, synthetic construct
  VARIANT 1..2: note = or R or H or any non-genetically coded or modified amino acid carrying a positive charge at pH 7.0
  VARIANT 5..6: note = or R or H or any non-genetically coded or modified amino acid carrying a positive charge at pH 7.0
  VARIANT 9: note = or R or H or any non-genetically coded or modified amino acid carrying a positive charge at pH 7.0
  VARIANT 3..4: note = W or a non-genetically coded lipophilic amino acid
  VARIANT 7..8: note = W or a non-genetically coded lipophilic amino acid
  VARIANT 1..9: note = three X positions are W and one is a non-genetically coded lipophilic amino acid
KKXXKKXXK

SEQ ID NO: 2 - protein, 9 aa, synthetic construct
  VARIANT 2..3: note = or R or H or any non-genetically coded or modified amino acid carrying a positive charge at pH 7.0
  VARIANT 6..7: note = or R or H or any non-genetically coded or modified amino acid carrying a positive charge at pH 7.0
  VARIANT 9: note = or R or H or any non-genetically coded or modified amino acid carrying a positive charge at pH 7.0
  VARIANT 1: note = W or a non-genetically coded lipophilic amino acid
  VARIANT 4..5: note = W or a non-genetically coded lipophilic amino acid
  VARIANT 8: note = W or a non-genetically coded lipophilic amino acid
  VARIANT 1..9: note = three X positions are W and one is a non-genetically coded lipophilic amino acid
XKKXXKKXK

SEQ ID NO: 3 - protein, 9 aa, synthetic construct
  VARIANT 1: note = or R or H or any non-genetically coded or modified amino acid carrying a positive charge at pH 7.0
  VARIANT 4..5: note = or R or H or any non-genetically coded or modified amino acid carrying a positive charge at pH 7.0
  VARIANT 8..9: note = or R or H or any non-genetically coded or modified amino acid carrying a positive charge at pH 7.0
  VARIANT 2..3: note = W or a non-genetically coded lipophilic amino acid
  VARIANT 6..7: note = W or a non-genetically coded lipophilic amino acid
  VARIANT 1..9: note = three X positions are W and one is a non-genetically coded lipophilic amino acid
KXXKKXXKK

SEQ ID NO: 4 - protein, 9 aa, synthetic construct
  VARIANT 1..2: note = or R or H or any non-genetically coded or modified amino acid carrying a positive charge at pH 7.0
  VARIANT 5: note = or R or H or any non-genetically coded or modified amino acid carrying a positive charge at pH 7.0
  VARIANT 8..9: note = or R or H or any non-genetically coded or modified amino acid carrying a positive charge at pH 7.0
  VARIANT 3..4: note = W or a non-genetically coded lipophilic amino acid
  VARIANT 6..7: note = W or a non-genetically coded lipophilic amino acid
  VARIANT 1..9: note = three X positions are W and one is a non-genetically coded lipophilic amino acid
KKXXKXXKK

SEQ ID NO: 5 - protein, 9 aa, synthetic construct
  VARIANT 1: note = or R or H or any non-genetically coded or modified amino acid carrying a positive charge at pH 7.0
  VARIANT 3..4: note = or R or H or any non-genetically coded or modified amino acid carrying a positive charge at pH 7.0
  VARIANT 7..8: note = or R or H or any non-genetically coded or modified amino acid carrying a positive charge at pH 7.0
  VARIANT 2: note = W or a non-genetically coded lipophilic amino acid
  VARIANT 5..6: note = W or a non-genetically coded lipophilic amino acid
  VARIANT 9: note = W or a non-genetically coded lipophilic amino acid
  VARIANT 1..9: note = three X positions are W and one is a non-genetically coded lipophilic amino acid
KXKKXXKKX

SEQ ID NO: 6 - protein, 9 aa, synthetic construct
  VARIANT 1..2: note = or R or H or any non-genetically coded or modified amino acid carrying a positive charge at pH 7.0
  VARIANT 5..6: note = or R or H or any non-genetically coded or modified amino acid carrying a positive charge at pH 7.0
  VARIANT 9: note = or R or H or any non-genetically coded or modified amino acid carrying a positive charge at pH 7.0
  SITE 3: note = a non-genetically coded lipophilic amino acid
KKXWKKWWK

SEQ ID NO: 7 - protein, 9 aa, synthetic construct
  VARIANT 1..2: note = or R or H or any non-genetically coded or modified amino acid carrying a positive charge at pH 7.0
  VARIANT 5..6: note = or R or H or any non-genetically coded or modified amino acid carrying a positive charge at pH 7.0
  VARIANT 9: note = or R or H or any non-genetically coded or modified amino acid carrying a positive charge at pH 7.0
  SITE 8: note = a non-genetically coded lipophilic amino acid
KKWWKKWXK

SEQ ID NO: 8 - protein, 9 aa, synthetic construct
  VARIANT 1..2: note = or R or H or any non-genetically coded or modified amino acid carrying a positive charge at pH 7.0
  VARIANT 5..6: note = or R or H or any non-genetically coded or modified amino acid carrying a positive charge at pH 7.0
  VARIANT 9: note = or R or H or any non-genetically coded or modified amino acid carrying a positive charge at pH 7.0
  SITE 4: note = a non-genetically coded lipophilic amino acid
KKWXKKWWK

SEQ ID NO: 9 - protein, 9 aa, synthetic construct
  VARIANT 2..3: note = or R or H or any non-genetically coded or modified amino acid carrying a positive charge at pH 7.0
  VARIANT 6..7: note = or R or H or any non-genetically coded or modified amino acid carrying a positive charge at pH 7.0
  VARIANT 9: note = or R or H or any non-genetically coded or modified amino acid carrying a positive charge at pH 7.0
  SITE 5: note = a non-genetically coded lipophilic amino acid
WKKWXKKWK

SEQ ID NO: 10 - protein, 9 aa, synthetic construct
  SITE 1: note = diphenylalanine
  MOD_RES 9: note = AMIDATION
XKKWWKKWK

SEQ ID NO: 11 - protein, 9 aa, synthetic construct
  SITE 5: note = diphenylalanine
  MOD_RES 9: note = AMIDATION
WKKWXKKWK

SEQ ID NO: 12 - protein, 9 aa, synthetic construct
  SITE 8: note = diphenylalanine
  MOD_RES 9: note = AMIDATION
WKKWWKKXK

SEQ ID NO: 13 - protein, 9 aa, synthetic construct
  SITE 1: note = biphenylalanine
  MOD_RES 9: note = AMIDATION
XKKWWKKWK

SEQ ID NO: 14 - protein, 9 aa, synthetic construct
  SITE 4: note = biphenylalanine
  MOD_RES 9: note = AMIDATION
WKKXWKKWK

SEQ ID NO: 15 - protein, 9 aa, synthetic construct
  SITE 5: note = D-diphenylalanine
  SITE 1: note = D-tryptophan
  SITE 2..3: note = D-lysine
  SITE 4: note = D-tryptophan
  SITE 6..7: note = D-lysine
  SITE 8: note = D-tryptophan
  SITE 9: note = D-lysine
  MOD_RES 9: note = AMIDATION
WKKWXKKWK

SEQ ID NO: 16 - protein, 9 aa, synthetic construct
  SITE 4: note = diphenylalanine
  MOD_RES 9: note = AMIDATION
KKWXKKWWK

SEQ ID NO: 17 - protein, 9 aa, synthetic construct
  SITE 4: note = diphenylalanine
  SITE 1..2: note = D-lysine
  SITE 5..6: note = D-lysine
  SITE 9: note = D-lysine
  MOD_RES 9: note = AMIDATION
KKWXKKWWK

SEQ ID NO: 18 - protein, 9 aa, synthetic construct
  SITE 4: note = diphenylalanine
  MOD_RES 9: note = AMIDATION
KKWXKKWXK

SEQ ID NO: 19 - protein, 9 aa, synthetic construct
  SITE 4: note = biphenylalanine
  MOD_RES 9: note = AMIDATION
KKWXKKWWK

SEQ ID NO: 20 - protein, 9 aa, synthetic construct
  SITE 2: note = biphenylalanine
  MOD_RES 9: note = AMIDATION
KXKKWWKKW

SEQ ID NO: 21 - protein, 9 aa, synthetic construct
  SITE 3: note = biphenylalanine
  MOD_RES 9: note = AMIDATION
KKXWKKWWK

SEQ ID NO: 22 - protein, 9 aa, synthetic construct
  SITE 7: note = diphenylalanine
  MOD_RES 9: note = AMIDATION
KKWWKKXWK

SEQ ID NO: 23 - protein, 9 aa, synthetic construct
  SITE 8: note = diphenylalanine
  MOD_RES 9: note = AMIDATION
KKWWKKWXK

SEQ ID NO: 24 - protein, 9 aa, synthetic construct
  SITE 3: note = diphenylalanine
  MOD_RES 9: note = AMIDATION
KWXKKWWKK

SEQ ID NO: 25 - protein, 9 aa, synthetic construct
  SITE 7: note = diphenylalanine
  MOD_RES 9: note = AMIDATION
KKWWKWXKK

SEQ ID NO: 26-27 - skipped (2 entries)

SEQ ID NO: 28 - protein, 9 aa, synthetic construct
  SITE 4: note = diphenylalanine
  MOD_RES 9: note = AMIDATION
RRWXRRWWR

SEQ ID NO: 29 - protein, 9 aa, synthetic construct
  SITE 6: note = diphenylalanine
  MOD_RES 9: note = AMIDATION
KWWKKXWKK

SEQ ID NO: 30 - protein, 9 aa, synthetic construct
  SITE 2: note = diphenylalanine
  MOD_RES 9: note = AMIDATION
KXKKWWKKW

SEQ ID NO: 31 - protein, 9 aa, synthetic construct
  SITE 3: note = diphenylalanine
  MOD_RES 9: note = AMIDATION
KKXWKKWWK

SEQ ID NO: 32 - protein, 9 aa, synthetic construct
  SITE 6: note = D-diphenylalanine
  SITE 1: note = D-lysine
  SITE 2..3: note = D-tryptophan
  SITE 4..5: note = D-lysine
  SITE 7: note = D-tryptophan
  SITE 8..9: note = D-lysine
  MOD_RES 9: note = AMIDATION
KWWKKXWKK

SEQ ID NO: 33 - protein, 9 aa, synthetic construct
  SITE 3: note = biphenylalanine
  MOD_RES 9: note = AMIDATION
RRXWRRWWR

SEQ ID NO: 34 - protein, 9 aa, synthetic construct
  SITE 3: note = diphenylalanine
  MOD_RES 9: note = AMIDATION
RRXWRRWWR

SEQ ID NO: 35 - protein, 9 aa, synthetic construct
  SITE 3: note = D-biphenylalanine
  SITE 1..2: note = D-lysine
  SITE 4: note = D-tryptophan
  SITE 5..6: note = D-lysine
  SITE 7..8: note = D-tryptophan
  SITE 9: note = D-lysine
  MOD_RES 9: note = AMIDATION
KKXWKKWWK

SEQ ID NO: 36 - protein, 9 aa, synthetic construct
  SITE 3: note = biphenylalanine
  SITE 1..2: note = D-lysine
  SITE 4: note = D-tryptophan
  SITE 5..6: note = D-lysine
  SITE 7..8: note = D-tryptophan
  SITE 9: note = D-lysine
  MOD_RES 9: note = AMIDATION
KKXWKKWWK

SEQ ID NO: 37 - protein, 9 aa, synthetic construct
  SITE 3: note = D-biphenylalanine
  MOD_RES 9: note = AMIDATION
KKXWKKWWK

SEQ ID NO: 38 - skipped

SEQ ID NO: 39 - protein, 9 aa, synthetic construct
  SITE 4: note = 1-naphthylalanine
  MOD_RES 9: note = AMIDATION
KKWXKKWWK

SEQ ID NO: 40 - protein, 9 aa, synthetic construct
  SITE 4: note = 2-naphthylalanine
  MOD_RES 9: note = AMIDATION
KKWXKKWWK

SEQ ID NO: 41 - protein, 9 aa, synthetic construct
  SITE 4: note = 2-amino-3-(anthracen-9-yl)propanoic acid
  MOD_RES 9: note = AMIDATION
KKWXKKWWK

SEQ ID NO: 42 - protein, 9 aa, synthetic construct
  SITE 4: note = 2-amino-3-[1,1':4',1"-terphenyl-4-yl]propionic acid
  MOD_RES 9: note = AMIDATION
KKWXKKWWK